(19) United States
(12) Patent Application Publication
(10) Pub. No.: US 2022/0193191 A1
(43) Pub. Date: Jun. 23, 2022

(54) LIPOCALIN MUTEIN FOR TREATMENT OF ASTHMA

(71) Applicant: AstraZeneca AB, Södertälje (SE)

(72) Inventors: Lena Therese AXELSSON, Södertälje (SE); David Robert CLOSE, Cambridge (GB); Philip GARDINER, Södertälje (SE); Aulikki Ingergard Alexandra JAUHIANINEN, Södertälje (SE); Ekaterina PARDALI, Södertälje (SE); Mary FITZGERALD, Hallbergmoos (DE); Gabriele MATSCHINER, Hallbergmoos (DE); Ingmar BRUNS, Boston, MA (US); Gunnel Marita OLSSON, Södertälje (SE)

(21) Appl. No.: 17/599,034

(22) PCT Filed: Mar. 25, 2020
(86) PCT No.: PCT/EP2020/058360

§ 371 Date: Sep. 28, 2021

Related U.S. Application Data
(60) Provisional application No. 62/826,791, filed on Mar. 29, 2019.
(60) Provisional application No. 62/845,774, filed on May 9, 2019.
(60) Provisional application No. 62/906,443, filed on Sep. 26, 2019.

(51) Int. Cl. A61K 38/17; A61K 9/00; A61P 11/06

(52) U.S. Cl. CPC A61K 38/1709; A61P 11/06; A61K 9/007

(57) ABSTRACT

The present invention relates to the treatment of asthma in a human subject by administering by inhalation a therapeutically effective amount of an anti-IL-4 receptor alpha (IL-4Ra) lipocalin mutein, or a variant or fragment thereof, to said subject, wherein the delivered dose of said lipocalin mutein, or variant or fragment thereof, is from about 0.1 mg to about 160 mg. The lipocalin mutein, or a variant or fragment thereof, may for example be administered at least once per day, once per day or twice per day.

### FIELD OF THE INVENTION

The present invention relates to the treatment of asthma in a human subject by administering by inhalation a therapeutically effective amount of an anti-IL-4 receptor alpha (IL-4Rα) lipocalin mutein, or a variant or fragment thereof, to said subject, wherein the delivered dose of said lipocalin mutein, or variant or fragment thereof, is from about 0.1 mg to about 160 mg. The lipocalin mutein, or a variant or fragment thereof, may for example be administered at least once per day, once per day or twice per day.

### BACKGROUND

Lipocalins are proteinaceous binding molecules that have antibody-like functions, which have naturally evolved to bind ligands. Lipocalins occur in many organisms, including vertebrates, insects, plants and bacteria. The members of the lipocalin protein family (Pervaiz, S., & Brew, K. (1987) FASEB J. 1, 209-214) are typically small, secreted proteins and have a single polypeptide chain. They are characterized by a range of different molecular-recognition properties: their ability to bind various, principally hydrophobic molecules (such as retinoids, fatty acids, cholesterols, prostaglandins, biliverdins, pheromones, tastants, and odorants), their binding to specific cell-surface receptors and their formation of macromolecular complexes. Although they have, in the past, been classified primarily as transport proteins, it is now clear that the lipocalins fulfil a variety of physiological functions. These include roles in retinol transport, olfaction, pheromone signalling, and the synthesis of prostaglandins. The lipocalins have also been implicated in the regulation of the immune response and the mediation of cell homoeostasis (reviewed, for example, in Flower, D. R. (1996) Biochem. J. 318, 1-14 and Flower, D. R. et al. (2000) Biochim. Biophys. Acta 1482, 9-24).

The lipocalins share unusually low levels of overall sequence conservation, often with sequence identities of less than 20%. In strong contrast, their overall folding pattern is highly conserved. The central part of the lipocalin structure consists of a single eight-stranded anti-parallel β-sheet closed back on itself to form a continuously hydrogen-bonded β-barrel. This β-barrel forms a central cavity. One end of the barrel is sterically blocked by the N-terminal peptide segment that runs across its bottom as well as three peptide loops connecting the β-strands. The other end of the β-barrel is open to solvent and encompasses a target-binding site, which is formed by four flexible peptide loops. It is this diversity of the loops in the otherwise rigid lipocalin scaffold that gives rise to a variety of different binding modes each capable of accommodating targets of different size, shape, and chemical character (reviewed, e.g., in Flower, D. R. (1996), supra; Flower, D. R. et al. (2000), supra, or Skerra, A. (2000) Biochim. Biophys. Acta 1482, 337-350).

Human tear lipocalin (TLPC or Tlc), also termed lipocalin-1, tear pre-albumin or von Ebner gland protein, was originally described as a major protein of human tear fluid (approximately one third of the total protein content), but has also been identified in several other secretory tissues including prostate, adrenal gland, thymus, mammary gland, testis, nasal mucosa and tracheal mucosa as well as corticotrophs of the pituitary gland. Homologous proteins have been found in rhesus monkey, chimpanzee, rat, mouse, pig, hamster, cow, dog and horse. Tear lipocalin is an unusual lipocalin member in that it exhibits an unusually broad ligand specificity, when compared to other lipocalins, and in its high promiscuity for relative insoluble lipids (see Redl, B. (2000) Biochim. Biophys. Acta 1482; 241-248). This feature of tear lipocalin has been attributed to the protein's function in inhibiting bacterial and fungal growth at the cornea. A remarkable number of lipophilic compounds of different chemical classes such as fatty acids, fatty alcohols, phospholipids, glycolipids and cholesterol are endogenous ligands of this protein. Interestingly, in contrast to other lipocalins, the strength of ligand (target) binding to tear lipocalin correlates with the length of the hydrocarbon tail for both alkyl amides and fatty acids. Thus, tear lipocalin binds most strongly to the least soluble lipids (Glasgow, B. J. et al. (1995) Curr. Eye Res. 14, 363-372; Gasymov, O. K. et al. (1999) Biochim. Biophys. Acta 1433, 307-320). The 1.8-A crystal structure of tear lipocalin revealed an unusually large cavity inside its β-barrel (Breustedt, D. A. et al. (2005) J. Biol. Chem. 280, 1, 484-493).

International patent application WO 99/16873 discloses polypeptides of the lipocalin family with mutated amino acid positions in the region of the four peptide loops, which are arranged at the end of the cylindrical β-barrel structure encompassing the binding pocket, and which correspond to those segments in the linear polypeptide sequence that includes the amino acid positions 28 to 45, 58 to 69, 86 to 99, and 114 to 129 of the bilin-binding protein of Pieris brassicae. Members of the lipocalin family have been reported to be post-translationally modified, e.g. phosphorylation and glycosylation of tear lipocalin (e.g. You, J., et al. (2010) Electrophoresis 31, 1853-1861). Nevertheless, no post-translational modification is required for their molecular recognition properties.

International patent application WO 00/75308 discloses muteins of the bilin-binding protein, which specifically bind digoxigenin, whereas international patent applications WO 03/029463 and WO 03/029471 relate to muteins of the human neutrophil gelatinase-associated lipocalin (hNGAL) and apolipoprotein D, respectively. In order to improve and fine tune ligand affinity, specificity, as well as folding stability, of a lipocalin variant further, various approaches using different members of the lipocalin family have been proposed (Skerra, A. (2001) Rev. Mol. Biotechnol. 74, 257-275; Schlehuber, S., and Skerra, A. (2002) Biophys. Chem. 96, 213-228), such as the replacement of additional amino acid residues. The PCT publication WO 2006/56464 discloses muteins of human neutrophil gelatinase-associated lipocalin with binding affinity for CTLA-4 in the low nanomolar range.

International patent application WO 2005/19256 discloses muteins of tear lipocalin with at least one binding site for different or the same target ligand and provides a method for the generation of such muteins of human tear lipocalin. According to this PCT application, certain amino acid stretches within the primary sequence of tear lipocalin, in particular the loop regions that include amino acids 7-14, 24-36, 41-49, 53-66, 69-77, 79-84, 87-98, and 103-110 of mature human tear lipocalin, are subjected to mutagenesis in order to generate muteins with binding affinities. The resulting muteins have binding affinities for the selected ligand (K_{D}) in the nanomolar range, in most cases >100 nM. International patent application WO 2008/015239 discloses muteins of tear lipocalin binding a given non-natural ligand, including the IL-4 receptor alpha. Binding affinities are in the nanomolar range. International patent application WO 2011/154420 describes high affinity muteins of human tear lipocalin that bind to human IL-4 receptor alpha in the nanomolar range and methods for producing such high affinity muteins. International patent application WO 2013/087660 describes the use of muteins of human tear lipocalin to treat disorders in which the IL-4/IL-13 pathway contributes to disease pathogenesis, including asthma.

### SUMMARY OF THE INVENTION

The present invention is based on in-human studies of an anti-IL-4 receptor alpha (IL-4Rα) human tear lipocalin, PRS-060/AZD1402, which is the first lipocalin-based treatment for asthma. The amino acid sequence of PRS-060/AZD1402 is shown in Table 20 as SEQ ID NO:1. PRS-060/AZD1402 antagonises the IL-4 receptor alpha (IL-4Rα) and is designed for inhalation. The first-in-human study in healthy subjects was conducted to assess the safety, tolerability and pharmacokinetics (PK) of inhaled single ascending doses and intravenous infusion (IV) doses. A second in-human study in subjects with mild asthma was conducted to assess the safety, tolerability and pharmacokinetics (PK) of inhaled multiple ascending doses. Following inhalation of AZD1402/PRS-060, systemic target engagement was determined by inhibition of IL-4 stimulated STAT6 phosphorylation (pSTAT6) and fractional exhaled nitric oxide (FeNO) a biomarker of lung inflammation, was measured as an indicator of local, pulmonary target engagement.

Based on the results of these studies, which are presented herein, the present invention provides a method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject, wherein the delivered dose of said lipocalin mutein, or variant or fragment thereof, is from about 0.1 mg to about 160 mg. The lipocalin mutein, or a variant or fragment thereof, may be administered at least once per day, once per day or twice daily.

The present invention further provides an anti-IL-4 receptor alpha (IL-4Rα) lipocalin mutein comprising the amino acid sequence set forth in SEQ ID NO: 1, or a variant or fragment thereof, for use in a method of treating asthma in a human subject, wherein the method comprises the step of administering said lipocalin mutein, or variant or fragment thereof, to said subject by inhalation, wherein the delivered dose of said lipocalin mutein, or variant or fragment thereof, is from about 0.1 mg to about 160 mg. The lipocalin mutein, or a variant or fragment thereof, may be administered at least once per day, once per day or twice daily.

In addition, the present invention provides the use of an anti-IL-4 receptor alpha (IL-4Rα) lipocalin mutein comprising the amino acid sequence set forth in SEQ ID NO: 1, or a variant or fragment thereof, for the manufacture of a medicament for use in treatment of asthma in a human subject, wherein the treatment comprises administering said lipocalin mutein, or variant or fragment thereof, to said subject by inhalation, wherein the delivered dose of said lipocalin mutein, or fragment or variant thereof, is from about 0.1 mg to about 160 mg. The lipocalin mutein, or a variant or fragment thereof, may be administered at least once per day, once per day or twice daily.

Based on the results of these studies, which are presented herein, the present invention provides a method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject at least once per day, wherein the delivered dose of said lipocalin mutein, is from about 0.1 mg to about 160 mg.

The present invention further provides an anti-IL-4 receptor alpha (IL-4Rα) lipocalin mutein comprising the amino acid sequence set forth in SEQ ID NO: 1, or a variant or fragment thereof, for use in a method of treating asthma in a human subject, wherein the method comprises the step of administering said lipocalin mutein, or variant or fragment thereof, to said subject by inhalation at least once per day, wherein the delivered dose of said lipocalin mutein is from about 0.1 mg to about 160 mg.

In addition, the present invention provides the use of an anti-IL-4 receptor alpha (IL-4Rα) lipocalin mutein comprising the amino acid sequence set forth in SEQ ID NO: 1, or a variant or fragment thereof, for the manufacture of a medicament for use in treatment of asthma in a human subject, wherein the treatment comprises administering said lipocalin mutein, or variant or fragment thereof, to said subject by inhalation at least once per day, wherein the delivered dose of said lipocalin mutein is from about 0.1 mg to about 160 mg.

In some embodiments, the delivered dose of said lipocalin mutein, or fragment or variant thereof, is from about 0.2 mg to about 60 mg. In some embodiments, the delivered dose of said lipocalin mutein, or fragment or variant thereof, is from about 0.6 mg to about 60 mg.

In some embodiments, the lipocalin mutein comprising the amino acid sequence set forth in SEQ ID NO: 1, or a variant or fragment thereof, is administered to said subject at least once per day. In some embodiments, said lipocalin mutein, or variant or fragment thereof, may be administered to the subject once daily. In some embodiments, said lipocalin mutein, or variant or fragment thereof, may be administered to the subject twice daily.

In some embodiments, the lipocalin mutein, or variant or fragment thereof may be administered to said subject for at least one day, for at least two days, for at least three days, for at least four days, for at least five days, for at least six days, for at least seven days, for at least eight days, for at least nine days or for at least ten days.

In some embodiments, the lipocalin mutein, or variant or fragment thereof may be administered to the subject twice daily for 9 days and once daily on the tenth day.

In some embodiments, based on the examples, the delivered dose of the lipocalin mutein, or variant or fragment thereof, is about 0.1 mg, about 0.5 mg, about 2 mg, about 8 mg, about 24 mg, about 72 mg or about 160 mg.

In some embodiments, based on the examples, the delivered dose of the lipocalin mutein, or variant or fragment thereof, is about 0.2 mg, about 2 mg, about 6 mg, about 20 mg or about 60 mg. In some embodiments, the delivered doses are administered at least once per day. In some embodiments, the delivered doses are administered once daily. In some embodiments, the delivered doses are administered twice daily.

In some embodiments, based on the examples, the delivered dose of the lipocalin mutein, or variant or fragment thereof, is about 0.2 mg, about 0.6 mg, about 2 mg, about 6 mg, about 20 mg or about 60 mg. In some embodiments, the delivered doses are administered at least once per day. In some embodiments, the delivered doses are administered once daily. In some embodiments, the delivered doses are administered twice daily.

In some embodiments, the delivered dose of the lipocalin mutein, or variant or fragment thereof, is sufficient to achieve systemic exposure, as shown in the examples. In some embodiments, the delivered dose of the lipocalin mutein, or variant or fragment thereof, does not result in a substantive portion of the inhaled lipocalin mutein entering the circulatory system or detectable systemic exposure, as shown in the examples.

In some embodiments, the delivered dose of the lipocalin mutein, or variant or fragment thereof, is at least about 8 mg. Systemic exposure of the lipocalin mutein was observed at delivered doses of at least about 8 mg, as reported herein.

In some embodiments, the delivered dose of the lipocalin mutein, or variant or fragment thereof, is at least about 6 mg. In some embodiments, the lipocalin mutein, or variant or fragment thereof is administered to the subject at least once per day. In some embodiments, the lipocalin mutein, or variant or fragment thereof is administered to the subject once per day. In some embodiments, the lipocalin mutein, or variant or fragment thereof is administered to the subject twice daily. Systemic exposure of the lipocalin mutein was observed at delivered doses of at least about 6 mg, as reported herein.

In other embodiments, the delivered dose of the lipocalin mutein, or variant or fragment thereof, is about 2 mg or less than about 2 mg. In some embodiments, the lipocalin mutein, or variant or fragment thereof is administered to the subject at least once per day. In some embodiments, the lipocalin mutein, or variant or fragment thereof is administered to the subject once per day. In some embodiments, the lipocalin mutein, or variant or fragment thereof is administered to the subject twice daily. In some embodiments, there is no substantive systemic exposure of the lipocalin mutein at delivered doses of about 2 mg or less than about 2 mg. In some embodiments, systemic exposure of the lipocalin mutein is not detectable at delivered doses of about 2 mg or less than about 2 mg. As reported herein, there was no detectable lipocalin mutein in the subjects' serum until 30 days post-dose when the delivered dose was less than about 2 mg and therefore, undetectable systemic exposure during this time.

In some embodiments, the delivered dose of the lipocalin mutein, or variant or fragment thereof, is about 0.6 mg or less than about 0.6 mg. In some embodiments, the lipocalin mutein, or variant or fragment thereof is administered to the subject at least once per day. In some embodiments, the lipocalin mutein, or variant or fragment thereof is administered to the subject once per day. In some embodiments, the lipocalin mutein, or variant or fragment thereof is administered to the subject twice daily. In some embodiments, there is no substantive systemic exposure of the lipocalin mutein at delivered doses of about 0.6 mg or less than about 0.6 mg. In some embodiments, systemic exposure of the lipocalin mutein is not detectable at delivered doses of about 0.6 mg or less than about 0.6 mg.

In some embodiments, the delivered dose of the lipocalin mutein, or variant or fragment thereof, is greater than about 0.6 mg and less than about 2 mg. In some embodiments, the lipocalin mutein, or variant or fragment thereof is administered to the subject at least once per day. In some embodiments, the lipocalin mutein, or variant or fragment thereof is administered to the subject once per day. In some embodiments, the lipocalin mutein, or variant or fragment thereof is administered to the subject twice daily.

In some embodiments, for example, when the delivered dose of the lipocalin mutein, or variant or fragment thereof, is at least about 8 mg, administering the lipocalin mutein, or variant or fragment thereof, to said subject results in inhibition of IL-4 stimulated STAT6 phosphorylation in CD3+ T cells in said subject.

In some embodiments, for example, when the delivered dose of the lipocalin mutein, or variant or fragment thereof, is at least about 6 mg, administering the lipocalin mutein, or variant or fragment thereof, to said subject results in inhibition of IL-4 stimulated STAT6 phosphorylation in CD3+ T cells in said subject. In other embodiments, for example, when the delivered dose of the lipocalin mutein, or variant or fragment thereof, is about 2 mg or less than about 2 mg, administering the lipocalin mutein, or variant or fragment thereof, to said subject does not result in a significant inhibition of IL-4 stimulated STAT6 phosphorylation in CD3+ T cells in said subject. In some embodiments, the lipocalin mutein, or variant or fragment thereof is administered to the subject at least once per day. In some embodiments, the lipocalin mutein, or variant or fragment thereof, is administered to the subject once per day. In some embodiments the lipocalin mutein, or variant or fragment thereof, is administered to the subject twice daily.

In specific embodiments, administering the lipocalin mutein, or variant or fragment thereof, may result in at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% inhibition of IL-4 stimulated STAT6 phosphorylation in CD3+ T cells in the subject. In an embodiment, administering the lipocalin mutein, or variant or fragment thereof, may result in at least about 20% inhibition of IL-4 stimulated STAT6 phosphorylation in CD3+ T cells in the subject. In other embodiments, administering the lipocalin mutein, or variant or fragment thereof, does not result in a significant inhibition of IL-4 stimulated STAT6 phosphorylation in CD3+ T cells in said subject, for example when the delivered dose of the lipocalin mutein, or variant or fragment thereof, is about 2 mg or less than about 2 mg.

In specific embodiments, administering the lipocalin mutein, or variant or fragment thereof, may result in at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% inhibition of IL-4 stimulated STAT6 phosphorylation in CD3+ T cells in the subject. In an embodiment, administering the lipocalin mutein, or variant or fragment thereof, may result in at least about 20% inhibition of IL-4 stimulated STAT6 phosphorylation in CD3+ T cells in the subject. In other embodiments, administering the lipocalin mutein, or variant or fragment thereof, does not result in a significant inhibition of IL-4 stimulated STAT6 phosphorylation in CD3+ T cells in said subject, for example when the delivered dose of the lipocalin mutein, or variant or fragment thereof, is about 0.6 mg or less than about 0.6 mg.

In any embodiments where the administration of the lipocalin mutein, or variant or fragment thereof, does not result in a significant inhibition of IL-4 stimulated STAT6 phosphorylation in CD3+ T cells in said subject, administering the lipocalin mutein, or variant or fragment thereof, may result in less than 10%, less than 5%, less than 4%, less than 3%, less than 2% of less than 1% inhibition of IL-4 stimulated STAT6 phosphorylation in CD3+ T cells in the subject.

Disclosed herein is a method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject, wherein the delivered dose of said lipocalin mutein results in inhibition of IL-4 stimulated STAT6 phosphorylation in CD3+ T cells in said subject. In some embodiments, the lipocalin mutein, or variant or fragment thereof, is administered to the subject at least once per day. In some embodiments, the lipocalin mutein, or variant or fragment thereof, is administered to the subject once per day. In some embodiments the lipocalin mutein, or variant or fragment thereof, is administered to the subject twice daily. In specific embodiments, administering the lipocalin mutein, or variant or fragment thereof, may result in at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% inhibition of IL-4 stimulated STAT6 phosphorylation in CD3+ T cells in the subject. In an embodiment, administering the lipocalin mutein, or variant or fragment thereof, may result in at least about 20% inhibition of IL-4 stimulated STAT6 phosphorylation in CD3+ T cells in the subject.

Also disclosed herein is a method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject, wherein the delivered dose of said lipocalin mutein does not result in significant inhibition of IL-4 stimulated STAT6 phosphorylation in CD3+ T cells in said subject. In some embodiments, the lipocalin mutein, or variant or fragment thereof, is administered to the subject at least once per day. In some embodiments, the lipocalin mutein, or variant or fragment thereof, is administered to the subject once per day. In some embodiments the lipocalin mutein, or variant or fragment thereof, is administered to the subject twice daily.

In any embodiments where the administration of the lipocalin mutein, or variant or fragment thereof, does not result in a significant inhibition of IL-4 stimulated STAT6 phosphorylation in CD3+ T cells in said subject, administering the lipocalin mutein, or variant or fragment thereof, may result in less than 10%, less than 5%, less than 4%, less than 3%, less than 2% of less than 1% inhibition of IL-4 stimulated STAT6 phosphorylation in CD3+ T cells in the subject.

In some embodiments, administering the lipocalin mutein, or variant or fragment thereof, may result in inhibition of IL-4 stimulated STAT6 phosphorylation in CD3+ T cells with an IC₅₀ of about 10 nM or lower, about 5 nM or lower, about 4 nM or lower, about 3 nM or lower, about 2 nM or lower, about 1 nM or lower, or about 0.5 nM or lower. In a specific embodiment, administering the lipocalin mutein, or variant or fragment thereof, inhibits IL-4 stimulated STAT6 phosphorylation in CD3+ T cells with an IC₅₀ of about 0.35 nM, as shown in FIG. 3. In a specific embodiment, administering the lipocalin mutein, or variant or fragment thereof, inhibits IL-4 stimulated STAT6 phosphorylation in CD3+ T cells with an IC₅₀ of about 0.306 nM, as shown in FIG. 11. In a specific embodiment, administering the lipocalin mutein, or variant or fragment thereof, inhibits IL-4 stimulated STAT6 phosphorylation in CD3+ T cells with an IC₅₀ of about 0.30 nM, as shown in FIG. 15.

As shown in Table 1, the lipocalin mutein having the amino acid sequence shown as SEQ ID NO:1 inhibits IL-4 stimulated STAT6 phosphorylation in CD3+ T cells in vitro with an IC₅₀ of about 1.3 nM.

Disclosed herein is a method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject, wherein the delivered dose of said lipocalin mutein results in inhibition of IL-4 stimulated STAT6 phosphorylation in CD3+ T cells with an IC₅₀ of about 10 nM or lower, about 5 nM or lower, about 4 nM or lower, about 3 nM or lower, about 2 nM or lower, about 1 nM or lower, or about 0.5 nM or lower. In some embodiments, the lipocalin mutein, or variant or fragment thereof, is administered to the subject at least once per day. In some embodiments, the lipocalin mutein, or variant or fragment thereof, is administered to the subject once per day. In some embodiments the lipocalin mutein, or variant or fragment thereof, is administered to the subject twice daily. In a specific embodiment, administering the lipocalin mutein, or variant or fragment thereof, inhibits IL-4 stimulated STAT6 phosphorylation in CD3+ T cells with an IC₅₀ of about 0.35 nM. In a specific embodiment, administering the lipocalin mutein, or variant or fragment thereof, inhibits IL-4 stimulated STAT6 phosphorylation in CD3+ T cells with an IC₅₀ of about 0.306 nM. In a specific embodiment, administering the lipocalin mutein, or variant or fragment thereof, inhibits IL-4 stimulated STAT6 phosphorylation in CD3+ T cells with an IC₅₀ of about 0.30 nM.

In any of the embodiments of the invention described herein, the lipocalin mutein, or fragment or variant thereof, may have a half-life (t_{1/2}) of from about 3 hours to about 7 hours in the subject following inhalation. These values are based on the data provided in Table 7, taking into account the standard deviation.

By way of comparison, following intravenous administration, the lipocalin mutein may have a half-life (t_{1/2}) of about 1.5 to 2.5 hours, based on the data shown in Table 8.

In any of the embodiments of the invention described herein, the peak serum concentration (Cₘₐₓ) of the lipocalin mutein following administration to the subject may be from about 6 ng/ml to about 400 ng/ml. These values are based on the data provided in Table 7 for cohorts 4-7, taking into account the standard deviation.

In any of the embodiments of the invention described herein, the serum concentration over time (AUC_{inf}) of said lipocalin mutein following administration to the subject is from about 60 h*ng/ml to about 5000 h*ng/ml. These values are based on the data provided in Table 7 for cohorts 4-7, taking into account the standard deviation.

Pharmacokinetic-related abbreviations (e.g. Cₘₐₓ and AUC_{inf}) and an explanation of their meanings are provided in Table 19 below.

In any of the embodiments of the invention described herein, fractional nitric oxide concentration in exhaled breath (FeNO) of the subject may be reduced following administration of said lipocalin mutein, or variant or fragment thereof, to said subject. In specific embodiments, FeNO may be reduced by at least 10%, by at least 15%, by at least 20%, by at least 25%, by at least 30%, by at least 35%, by at least 40%, by at least 45% or by at least 50% compared to a control subject following administration of said lipocalin mutein, or variant or fragment thereof, to said subject, wherein the control subject is a human patient who has not been administered said lipocalin mutein, or variant or fragment thereof. The control subject may be the same subject (with FeNO being assessed prior to administration of a lipocalin mutein) or a different subject who has not been administered any lipocalin mutein. In one embodiment, the control subject may have received a placebo. A suitable placebo may comprise a physiologically buffered salt solution, such as the solution used to formulate the lipocalin mutein, for example a phosphate buffered saline solution.

The data presented herein demonstrate that FeNO may be reduced even when there is no detectable systemic exposure of said lipocalin mutein in the (treated) subject's serum. This may indicate that a reduction in local inflammation may be achieved, as assessed using FeNO as a biomarker, without detectable systemic exposure of the lipocalin mutein. Therefore, a delivered dose of the lipocalin mutein, or variant or fragment thereof, of about 2 mg or less than about 2 mg may result in a reduction of FeNO as a result of local lung exposure, without a substantive portion of the inhaled lipocalin mutein entering the circulatory system or detectable systemic exposure. Thus, a delivered dose of the lipocalin mutein, or variant or fragment thereof, of about 2 mg or less than about 2 mg may provide clinical benefit to a human asthma patient. Therefore, a delivered dose of the lipocalin mutein, or variant or fragment thereof, of about 0.6 mg or less than about 0.6 mg may result in a reduction of FeNO as a result of local lung exposure, without a substantive portion of the inhaled lipocalin mutein entering the circulatory system or detectable systemic exposure. Thus, a delivered dose of the lipocalin mutein, or variant or fragment thereof, of about 0.6 mg or less than about 0.6 mg may provide clinical benefit to a human asthma patient.

In any of the embodiments of the invention described herein, the lipocalin mutein, or variant or fragment thereof, may be administered to the subject by nebulisation.

When the lipocalin mutein, or variant or fragment thereof, is administered by nebulisation, the nominal or metered dose (which is the dose of lipocalin mutein in the nebuliser) is from about 0.25 mg to about 400 mg. This is the nominal or metered dose present in the InnoSpire Go nebulizer (Philips) used in the examples described herein. The person skilled in the art would know that different devices are available for administration by inhalation, as described herein, and would be readily able to determine the delivered dose in accordance with the invention based on the nominal or metered dose in the particular device used to administer the lipocalin mutein.

In some embodiments, the nominal dose of the lipocalin mutein, or variant or fragment thereof, is at least about 20 mg. Systemic exposure of the lipocalin mutein was observed at nominal doses of at least about 20 mg, as reported herein, and administering the lipocalin mutein, or variant or fragment thereof, to said subject results in inhibition of IL-4 stimulated STAT6 phosphorylation in CD3+ T cells in said subject.

In some embodiments, the nominal dose of the lipocalin mutein, or variant or fragment thereof, is at least about 15 mg. In some embodiments, the lipocalin mutein, or variant or fragment thereof, is administered to the subject at least once per day. In some embodiments, the lipocalin mutein, or variant or fragment thereof, is administered to the subject once per day. In some embodiments, the lipocalin mutein, or variant or fragment thereof, is administered to the subject twice daily. Systemic exposure of the lipocalin mutein was observed at nominal doses of at least about 15 mg, as reported herein, and administering the lipocalin mutein, or variant or fragment thereof, to said subject results in inhibition of IL-4 stimulated STAT6 phosphorylation in CD3+ T cells in said subject.

In other embodiments, the nominal dose of the lipocalin mutein, or variant or fragment thereof, is about 5 mg or less than about 5 mg. In some embodiments, the lipocalin mutein, or variant or fragment thereof, is administered to the subject at least once per day. In some embodiments, the lipocalin mutein, or variant or fragment thereof, is administered to the subject once per day. In some embodiments, the lipocalin mutein, or variant or fragment thereof, is administered to the subject twice daily. In some embodiments, there is no substantive systemic exposure of the lipocalin mutein at nominal doses of about 5 mg or less than about 5 mg. In some embodiments, systemic exposure of the lipocalin mutein is not detectable at nominal doses of about 5 mg or less than about 5 mg. As reported herein, there was no detectable lipocalin mutein in the (treated) subjects' serum measured for 30 days post-dose when the nominal dose was less than about 5 mg and therefore, undetectable systemic exposure during this time.

In some embodiments, the nominal dose of the lipocalin mutein, or variant or fragment thereof, is about 1.5 mg or less than about 1.5 mg. In some embodiments, the lipocalin mutein, or variant or fragment thereof, is administered to the subject at least once per day. In some embodiments, the lipocalin mutein, or variant or fragment thereof, is administered to the subject once per day. In some embodiments, the lipocalin mutein, or variant or fragment thereof, is administered to the subject twice daily. In some embodiments, there is no substantive systemic exposure of the lipocalin mutein at nominal doses of about 1.5 mg or less than about 1.5 mg. In some embodiments, systemic exposure of the lipocalin mutein is not detectable at nominal doses of about 1.5 mg or less than about 1.5 mg. As reported herein, there was no detectable lipocalin mutein in the (treated) subjects' serum measured for 30 days post-dose when the nominal dose was less than about 1.5 mg and therefore, undetectable systemic exposure during this time.

In some embodiments, the nominal dose of the lipocalin mutein, or variant or fragment thereof, is greater than about 1.5 mg and less than about 5 mg. In some embodiments, the lipocalin mutein, or variant or fragment thereof, is administered to the subject at least once per day. In some embodiments, the lipocalin mutein, or variant or fragment thereof, is administered to the subject once per day. In some embodiments, the lipocalin mutein, or variant or fragment thereof, is administered to the subject twice daily.

### SUMMARY OF THE FIGURES

Embodiments and experiments illustrating the principles of the invention will now be discussed with reference to the accompanying figures in which:

FIG. 1 shows that the in vitro addition of a lipocalin mutein specific for IL-4Rα (PRS-060/AZD1402 having SEQ ID NO: 1) inhibits IL-4 signalling in whole blood, reducing levels of STAT6 phosphorylation (pSTAT6) (FIG. 1A) and eotaxin-3 (FIG. 1B), TARC (FIG. 10), and MDC (FIG. 1D) production induced by IL-4 stimulation. The lipocalin mutein has similar potency to a reference IL-4Rα antibody (Dupilumab) in these functional in vitro assays. Dupilumab is a fully human Ig4 monoclonal antibody directed against the interleukin-4 receptor subunit a (IL-4Rα) of IL-4 and IL-13 receptors. It is normally given by subcutaneous injection and is approved for the treatment of atopic dermatitis and moderate to severe eosinophilic asthma in the US.

FIG. 2 shows the ex vivo inhibition of STAT6 phosphorylation (pSTAT6) in whole blood stimulated with IL-4 of subjects receiving inhaled PRS-060/AZD1402 (having SEQ ID NO: 1) at different delivered doses.

FIG. 3 shows ex vivo inhibition of STAT6 phosphorylation (pSTAT6) in whole blood stimulated with IL-4 of subjects receiving inhaled PRS-060/AZD1402 (having SEQ ID NO: 1). A dose-dependent inhibition of STAT6 phosphorylation was observed, with an IC₅₀ value of 0.35 nM.

FIG. 4 provides the result of pharmacokinetic analyses of single dose of PRS-060/AZD1402 (shown as SEQ ID NO: 1) administered by oral inhalation in healthy subjects. Systemic exposure of inhaled PRS-060/AZD1402 was observed at a delivered dose of 8.00 mg or higher. Mean serum PRS-060/AZD1402 concentrations increased with the escalating doses. A slow decline in serum PK following inhalation was observed, indicating absorption-driven elimination. This figure shows the mean (SD) serum concentration of inhaled PRS-060/AZD1402 versus the time profiles in cohorts 4 to 7 only with a linear scale (PK population). SD=standard deviation; PK=pharmacokinetics.

FIG. 5 provides the result of pharmacokinetic analyses of single dose of PRS-060/AZD1402 (shown as SEQ ID NO: 1) administered by oral inhalation in healthy subjects. Systemic exposure of inhaled PRS-060/AZD1402 was observed at a delivered dose of 8.00 mg or higher. Mean serum PRS-060/AZD1402 concentrations increased with the escalating doses. A slow decline in serum PK following inhalation was observed, indicating absorption-driven elimination. This figure shows the mean (SD) serum concentration of inhaled PRS-060/AZD1402 versus the time profiles in cohorts 4 to 7 only with a log-linear scale (PK population). SD=standard deviation; PK=pharmacokinetics.

FIG. 6 provides the result of pharmacokinetic analyses of single dose of PRS-060/AZD1402 (shown as SEQ ID NO: 1) administered by intravenous administration in healthy subjects. Mean serum levels of PRS-060/AZD1402 indicated a rapid elimination phase with t_{1/2} of approximately half that observed in subjects that received inhaled doses. This figure shows the mean (SD) serum concentration of PRS-060/AZD1402 versus the time profiles following intravenous administration in cohort 8 (1 mg) and cohort 9 (2 mg) with a linear scale (PK population). SD=standard deviation; PK=pharmacokinetics.

FIG. 7 provides the result of pharmacokinetic analyses of single dose of PRS-060/AZD1402 (shown as SEQ ID NO: 1) administered by intravenous administration in healthy subjects. Mean serum levels of PRS-060/AZD1402 indicated a rapid elimination phase with t_{1/2} of approximately half that observed in subjects that received inhaled doses. This figure shows the mean (SD) serum concentration of PRS-060/AZD1402 versus the time profiles following intravenous administration in cohort 8 (1 mg) and cohort 9 (2 mg) with a log-linear scale (PK population). SD=standard deviation; PK=pharmacokinetics.

FIG. 8 shows the mean percentage change of fractional nitric oxide concentration in exhaled breath (FeNO) from baseline for placebo group and delivered doses 2 mg, 6 mg and 20 mg. Group means are calculated based on log(FeNO) change from baseline, back-transformed to linear scale and expressed as percentage.

FIG. 9 shows the serum mean exposure profiles after twice-daily delivered doses of 2, 6 and 20 mg PRS-060/AZD1402.

FIG. 10 shows the ex vivo inhibition of STAT6 phosphorylation (pSTAT6) in whole blood stimulated with IL-4 of subjects receiving inhaled PRS-060/AZD1402 (having SEQ ID NO: 1) at different delivered doses (2.0 mg, 6.0 mg and 20 mg).

FIG. 11 shows the ex vivo inhibition of STAT6 phosphorylation (pSTAT6) in whole blood stimulated with IL-4 of subjects receiving inhaled PRS-060/AZD1402 (having SEQ ID NO: 1). A dose-dependent inhibition of STAT6 phosphorylation was observed, with an IC50 value of 0.306 nM.

FIG. 12 shows the mean percentage FeNO change relative to baseline for placebo group (n=12) and dose groups of cohort 1-4. Group means are calculated based on log(FeNO) change from baseline, back-transformed to linear scale and expressed as percentage.

FIG. 13 shows the serum median exposure profiles after twice-daily delivered doses of 2, 6, 20 and 60 mg PRS-060/AZD1402.

FIG. 14 shows the ex vivo inhibition of STAT6 phosphorylation (pSTAT6) in whole blood stimulated with IL-4 of subjects receiving inhaled PRS-060/AZD1402 (having SEQ ID NO: 1) at different delivered doses (2.0 mg, 6.0 mg, 20 mg and 60 mg).

FIG. 15 shows ex vivo inhibition of STAT6 phosphorylation (pSTAT6) in whole blood stimulated with IL-4 of subjects receiving inhaled PRS-060/AZD1402 (having SEQ ID NO: 1). A dose-dependent inhibition of STAT6 phosphorylation was observed, with an IC50 value of 0.30 nM.

FIG. 16 shows the MAD study design corresponding only to cohorts 1˜4 of Example 4. Doses shown are multiple device doses (delivered doses b.i.d. (twice daily)) of PRS-060/AZD1402. b.i.d. doses administered 12 hours apart. On day −1, 1 day before receiving the first dose of AZD1402/PRS-060 or matching placebo, participants were evaluated to confirm eligibility. Participants checked into the hospital/study site and remained in the hospital/study site until checkout 48 hours after (day 12) the last dose of the study medication (day 10). Study medication was administered using an InnoSpire Go nebulizer at delivered doses between 2 mg and 60 mg b.i.d. for 9 days with one dose on day 10. The study duration from screening to post-study follow-up visit was approximately 9 weeks for each participant.

FIG. 17 shows the SAD study design of Example 2.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present invention relates to a method of treating asthma in a human subject. Asthma is a chronic, complex and heterogeneous respiratory disease characterised by a range of pathogenic features including pulmonary inflammation, mucus hypersecretion, variable airway obstruction and airway remodelling. It is defined by a history of respiratory symptoms that include wheezing, shortness of breath and cough which vary over time and in severity. Both symptoms and airway obstruction can be triggered by a range of factors including exercise, exposure to inhaled irritants or allergens or respiratory infections. Patients are at risk of worsening of their asthma (exacerbations). These exacerbations of asthma can be life threatening and can significantly impact the patient's quality of life. The treatment for most asthma patients, consists of a treatment regime of a controller and bronchodilator therapy. Inhaled corticosteroids (ICS) are considered the “gold standard” in controlling asthma symptoms and long acting beta-agonists (LABA) are the most effective bronchodilators currently available. Oral corticosteroids remain standard of care in severe asthma but are associated with significant side-effects, whilst omalizumab, an anti-IgE monoclonal antibody; benralizumab, mepolizumab and reslizumab, antilL-5 antibodies, and dupilumab (US) a monoclonal antibody blocker of IL-4Rα and IL-13 offer a limited number of options for the severe patients. Additionally, patients frequently remain uncontrolled on ICS/LABA and even the limited number of alternative therapies, highlighting an important unmet need.

Interleukin-4, interleukin-13, interleukin-4-receptor alpha and the signal transducer and activator of transcription factor-6 are key components in the development of airway inflammation, mucus production, and airway hyper-responsiveness in asthma.

The method of treating asthma comprises administering a therapeutically effective amount of an anti-IL-4 receptor alpha (IL-4Rα) lipocalin mutein, or a variant or fragment thereof, comprising the amino acid sequence set forth in SEQ ID NO: 1.

By “therapeutically effective amount” it is meant a dose that produces the effects for which it is administered. A “therapeutically effective amount” of a lipocalin mutein as described herein may vary according to factors such as age, body weight, general health, sex, diet, time of administration, drug interaction and the severity of the condition may be necessary, and will be ascertainable with routine experimentation by those skilled in the art. A therapeutically effective amount, when used in the present application, is also one in which any toxic or detrimental effects of the lipocalin mutein are outweighed by the therapeutically beneficial effects.

Interleukin-4 receptor alpha chain (IL-4Rα) is a type I transmembrane protein that can bind interleukin 4 and interleukin 13 to regulate IgE antibody production in B cells. Among T cells, the encoded protein also can bind interleukin 4 to promote differentiation of Th2 cells.

Lipocalin muteins that are specific for IL-4 receptor alpha (IL-4Rα), in particular human IL-4Rα are disclosed in International patent publications WO 2008/015239, WO 2011/154420, and WO 2013/087660. Human interleukin-4 receptor alpha chain may have the amino acid sequence of SWISS PROT Data Bank Accession No. P24394, which is shown as SEQ ID NO:4, or of fragments thereof. An illustrative example of a fragment of human interleukin-4 receptor alpha chain includes amino acids 26 to 232 of IL-4 receptor alpha.

The IL-4Rα specific lipocalin mutein having the amino acid sequence shown as SEQ ID NO:1 is a mutein of human tear lipocalin.

As used herein, a “mutein” refers to the exchange, deletion, or insertion of one or more nucleotides or amino acids, compared to the naturally occurring (wild-type) nucleic acid or protein “reference” scaffold, which is preferably mature human tear lipocalin shown as SEQ ID NO: 3. Said “reference scaffold” also includes mutein, or fragment or variant thereof, as described herein.

The amino acid sequence of human tear lipocalin is provided by SWISS-PROT Data Bank Accession Number P31025, as shown in SEQ ID NO: 2. Mature human tear lipocalin does not include the N-terminal signal peptide that is included in the sequence of SWISS-PROT Accession Number P31025, i.e. it lacks the N-terminal signal peptide (amino acids 1-18) that is included in the sequence of SWISS-PROT Accession Number P31025. The amino acid sequence of mature human tear lipocalin is shown in SEQ ID NO:3.

The lipocalin mutein used in the present invention comprises SEQ ID NO:1 or is a variant or fragment thereof. The lipocalin mutein shown as SEQ ID NO:1 is a variant of mature human tear lipocalin shown as SEQ ID NO:3, which lacks the first four amino acids and includes inter alia the following amino acid substitutions at the positions corresponding to the sequence positions of the amino acid sequence of mature human tear lipocalin shown as SEQ ID NO: 3: Arg 26→Ser, Glu 27→Arg, Phe 28→Cys, Glu 30→Arg, Met 31→Ala, Asn 32→Val, Leu 33→Tyr, Glu 34→Asn, Met 55→Ala, Leu 56→Gln, Ile 57→Arg, Ser 58→Lys, Cys 61→Trp, Glu 63→Lys, Asp 80→Ser, Lys 83→Arg, Glu 104→Leu, Leu 105→Cys, His 106→Pro and Lys 108→Gln.

The lipocalin mutein used in the present invention comprises SEQ ID NO:1 or is a variant or fragment thereof. The lipocalin mutein shown as SEQ ID NO:1 is a variant of mature human tear lipocalin shown as SEQ ID NO:3, which lacks the first four amino acids and includes inter alia the following amino acid substitutions at the positions corresponding to the sequence positions of the amino acid sequence of mature human tear lipocalin shown as SEQ ID NO: 3: Arg 26→Ser, Glu 27→Arg, Phe 28→Cys, Glu 30→Arg, Met 31→Ala, Asn 32→Val, Leu 33→Tyr, Glu 34→Asn, Val 53→Phe, Met 55→Ala, Leu 56→Gln, Ile 57→Arg, Ser 58→Lys, Cys 61→Trp, Glu 63→Lys, Val 64→Tyr, Ala 66→Leu, Asp 80→Ser, Lys 83→Arg, Tyr 100→His, Cys 101→Ser, Glu 104→Leu, Leu 105→Cys, His 106→Pro, Lys 108→Gln, Arg 111→Pro, Lys 114→Trp and Cys 153→Ser.

As used herein, the term “variant” relates to derivatives of a protein or polypeptide that include mutations, for example by substitutions, deletions, insertions, and/or chemical modifications of an amino acid sequence or nucleotide sequence. In some embodiments, such mutations and/or chemical modifications do not reduce the functionality of the protein or peptide. Such substitutions may be conservative, i.e., an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. Such variants include proteins or polypeptides, wherein one or more amino acids have been substituted by their respective D-stereoisomers or by amino acids other than the naturally occurring 20 amino acids, such as, for example, ornithine, hydroxyproline, citrulline, homoserine, hydroxylysine, norvaline. Such variants also include, for instance, proteins or polypeptides in which one or more amino acid residues are added or deleted at the N- and/or C-terminus such as a deletion of four amino acids from the N-terminus and/or two amino acids from the C-terminus. Generally, a variant has at least about 50%, at least about 60%, at least about 70%, at least about 75%, at least about 80%, at least about 85%, at least about 90%, at least about 92%, at least about 95%, at least about 96%, at least about 97%, at least about 98%, or at least about 99% amino acid sequence identity with the native sequence protein or polypeptide. A variant preferably retains the biological activity, e.g. binding the same target, of the protein or polypeptide from which it is derived.

Thus, a variant of the lipocalin mutein comprising the amino acid set forth in SEQ ID NO:1 in accordance with the present invention has at least about 50%, at least about 60%, at least about 70%, at least about 75%, at least about 80%, at least about 85%, at least about 90%, at least about 92%, at least about 95%, at least about 96%, at least about 97%, at least about 98%, or at least about 99% amino acid sequence identity with the amino acid sequence shown as SEQ ID NO:1 and retains the ability to bind to IL-4 receptor alpha, in particular human IL-4Rα, or a fragment thereof. Preferably, the variant of the lipocalin mutein is capable of inhibiting IL-4 from binding to IL-4Rα.

In some embodiments, a variant of the lipocalin mutein comprising the amino acid set forth in SEQ ID NO:1 in accordance with the present invention has at least about 50%, at least about 60%, at least about 65%, at least about 70%, at least about 72%, at least about 74%, at least about 75%, at least about 76%, at least about 77%, at least about 79%, at least about 80%, at least about 85%, at least about 90%, at least about 92%, at least about 95%, at least about 96%, at least about 97%, at least about 98%, or at least about 99% amino acid sequence identity with the amino acid sequence of mature human tear lipocalin, shown as SEQ ID NO:3 and retains the ability to bind to IL-4 receptor alpha, in particular human IL-4Rα, or a fragment thereof. Preferably, the variant of the lipocalin mutein is capable of inhibiting IL-4 from binding to IL-4Rα.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question —with respect to the number of residues in the longer of these two sequences. Sequence identity is measured by dividing the number of identical amino acid residues by the total number of residues and multiplying the product by 100.

A skilled artisan will recognize available computer programs, for example BLAST (Altschul et al., Nucleic Acids Res, 1997), BLAST2 (Altschul et al., J Mol Biol, 1990), FASTA (which uses the method of Pearson and Lipman (1988)), the TBLASTN program, of Altschul et al. (1990) supra, GAP (Wisconsin GCG package, Accelerys Inc, San Diego USA) and Smith-Waterman (Smith and Waterman, J Mol Biol, 1981), for determining sequence identity using standard parameters. The percentage of sequence identity can, for example, be determined herein using the program BLASTP, version 2.2.5, Nov. 16, 2002 (Altschul et al., Nucleic Acids Res, 1997). In this embodiment, the percentage of homology is based on the alignment of the entire protein or polypeptide sequences (matrix: BLOSUM 62; gap costs: 11.1; cut off value set to 10⁻³) including the polypeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment. Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps, which are spaces in an alignment that are the result of additions or deletions of amino acids. Generally, default parameters are used, with a gap creation penalty equaling 12 and a gap extension penalty equaling 4.

Specifically, in order to determine whether an amino acid residue of the amino acid sequence of a lipocalin (mutein) is different from a lipocalin mutein having the amino acid sequence shown as SEQ ID NO:1, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments. Accordingly, SEQ ID NO:1 can serve as “reference sequence”, while the amino acid sequence of a lipocalin different from the lipocalin mutein having the amino acid sequence shown as SEQ ID NO:1 described herein serves as “query sequence”.

The term “fragment” as used herein in connection with the lipocalin muteins of the disclosure relates to proteins or peptides derived from the lipocalin mutein comprising the amino acid sequence set forth in SEQ ID NO:1 that are N-terminally and/or C-terminally truncated, i.e. lacking at least one of the N-terminal and/or C-terminal amino acids. Such a fragment may lack up to 1, up to 2, up to 3, up to 4, up to 5, up to 10, up to 15, up to 20, up to 25, or up to 30 (including all numbers in between) of the N-terminal and/or C-terminal amino acids. As an illustrative example, such a fragment may lack the one, two, three, or four N-terminal and/or one or two C-terminal amino acids. It is understood that the fragment is preferably a functional fragment of a full-length lipocalin (mutein), which means that it preferably comprises the binding pocket of the full length lipocalin (mutein) from which it is derived. As an illustrative example, such a functional fragment may comprise at least amino acids at positions 5-158, 1-156, 5-156, 5-153, 26-153, 5-150, 9-148, 12-140, 20-135, or 26-133 corresponding to the linear polypeptide sequence of mature human tear lipocalin. Such fragments may include at least 10, at least 20, at least 30, at least 40, at least 50, at least 60, at least 70, at least 80, at least 90 or at least at least 100 consecutive amino acids of the sequence shown as SEQ ID NO: 1 and are usually detectable in an immunoassay of the lipocalin mutein having the amino acid sequence SEQ ID NO:1. A fragment may have at least about 50%, at least about 60%, at least about 70%, at least about 75%, at least about 80%, at least about 85%, at least about 90%, at least about 92%, at least about 95%, at least about 96%, at least about 97%, at least about 98%, or at least about 99% amino acid sequence identity with the amino acid sequence shown as SEQ ID NO:1. Preferably the fragment retains the ability to bind to IL-4 receptor alpha, in particular human IL-4Rα, or to a fragment thereof. Preferably, the fragment of the lipocalin mutein is capable of inhibiting IL-4 from binding to IL-4Rα.

A “fragment” with respect to the corresponding target IL-4Rα of the disclosure (which is described in UniProt P24394 and shown as SEQ ID NO: 4, which does not include the 25-residue signal peptide) refers to N-terminally and/or C-terminally truncated IL-4Rα or protein domains of IL-4Rα. Fragments of IL-4Rα as described herein retain the capability of the full-length IL-4Rα to be recognized and/or bound by a lipocalin mutein of the disclosure. As an illustrative example, the fragment may be an extracellular domain of IL-4Rα, such as an extracellular domain comprising amino acid residues 26-232 of UniProt P24394, which is shown as SEQ ID NO:5.

The lipocalin mutein according to the present invention is administered to the human subject by inhalation. As used herein, administration by inhalation refers to administration of the lipocalin mutein, usually by oral inhalation. The lipocalin mutein may be in the form of a nebulised liquid aerosol, or a liquid spray. The lipocalin mutein may be administered by nebulisation.

Means and devices for inhaled administration of the lipocalin mutein are known to the skilled person. Such means and devices include nebulizers and non-pressurised metered dose inhalers. Other means and devices suitable for directing inhaled administration of a lipocalin mutein are also known in the art.

A nebulizer is a drug delivery device used to administer medication in the form of a mist inhaled into the lungs. Different types of nebulizers are known to the skilled person and include jet nebulizers, ultrasonic wave nebulizers and vibrating mesh technology. Some nebulizers provide a continuous flow of nebulized solution, i.e. they will provide continuous nebulization over a long period of time, regardless of whether the subject inhales from it or not, while others are breath-actuated, i.e. the subject only gets some dose when they inhale from it.

A non-pressurised metered-dose inhaler (MDI), also known as a soft mist inhaler, is a device that delivers a specific amount of medication to the lungs, in the form of a short burst of liquid aerosolized medicine. Such a metered-dose inhaler commonly consists of three major components; a canister which comprises the formulation to be administered, a metering valve, which allows a metered quantity of the formulation to be dispensed with each actuation, and an actuator (or mouthpiece) which allows the patient to operate the device and directs the liquid aerosol into the patient's lungs.

Lipocalin muteins for use in the present invention will usually be administered in the form of a pharmaceutical composition, which may comprise at least one component in addition to the specific binding member. Thus, pharmaceutical compositions for use in accordance with the present invention may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. For example, the lipocalin mutein for use in accordance with the present invention may be formulated in an aqueous solution of phosphate buffered saline (PBS).

The pharmaceutical composition comprising the lipocalin mutein may be administered alone or in combination with other treatments, either simultaneously or sequentially.

In the method for treating asthma disclosed herein, the delivered dose of said lipocalin mutein is from about 0.1 mg to about 160 mg. A “delivered dose” refers to the dose of lipocalin mutein that is delivered to a subject, i.e. the dose that comes out of an inhalation device when applying the device. For example, nebulizers are sometimes intentionally overfilled as the final total volume will not be nebulised. For a nebulizer, a delivered dose is commonly less than 50% of the nominal dose, which is the dose of lipocalin mutein loaded into the device. The nominal dose is also known as the metered dose. A skilled person can easily determine a delivered dose by determining the amount of lipocalin mutein that comes out of the inhalation device. For example, methods used to measure the “delivered dose” experimentally are provided in section 2.9.44 of the European Pharmacopeia 9.0.

The nominal (or metered) doses of 0.25 mg, 1.25 mg, 5 mg, 20 mg, 60 mg, 180 mg and 400 mg loaded into the nebulizer in the SAD study (Example 2) described herein correlate to delivered doses of 0.1 mg, 0.5 mg, 2.0 mg, 8.0 mg, 24 mg, 72 mg and 160 mg respectively.

The nominal (or metered) doses of 0.5 mg, 5 mg, 15 mg, 50 mg and 150 mg loaded into the nebulizer and administered twice daily in the MAD study (Example 3 and Example 4) described herein correlate to delivered doses of 0.2 mg, 2.0 mg, 6.0 mg, 20 mg and 60 mg respectively administered twice daily. A nominal or metered dose of 1.5 mg correlates to a delivered dose of 0.6 mg.

Results from the SAD study (Example 2) presented herein indicate that systemic exposure occurs at a delivered dose of at least about 8 mg of the lipocalin mutein, whereas at delivered doses below about 2 mg, no detectable systemic exposure is observed.

Results from cohorts 1-3 of the MAD study (Example 3) presented herein indicate that systemic exposure occurs at a delivered dose of at least about 6 mg of the lipocalin mutein, whereas at delivered doses about 2 mg or below about 2 mg, no detectable systemic exposure is observed.

Results from cohorts 1-5 of the MAD study (Example 4) presented herein indicate that systemic exposure occurs at a delivered dose of at least about 6 mg of the lipocalin mutein, whereas at delivered doses about 2 mg or below about 2 mg, no detectable systemic exposure is observed.

As used herein “systemic exposure” means that a substantive portion of the inhaled lipocalin mutein enters the circulatory system and, optionally, that the entire body may be affected by the lipocalin mutein. Systemic exposure may mean that the amount of the lipocalin mutein that enters the circulatory system is quantifiable. Systemic exposure may equate to the concentration of lipocalin mutein that enters the bloodstream that is quantifiable. This exposure can be represented by the blood (serum, plasma or whole blood) concentration of the lipocalin mutein which can be measured over time and recorded by a range of parameters including the area under the curve (AUC). Systemic exposure to lipocalin mutein can also impact biomarkers, the levels of which can correlate directly to concentration of lipocalin mutein and therefore to systemic exposure. The term “quantifiable” or “detectable,” when used in connection with systemic exposure, refers to the exposure represented by the blood (serum, plasma or whole blood) concentration of the lipocalin mutein or by the levels of biomarkers measurable by one or more analytical methods known in art. Such analytical methods include, but are not limited to, ELISA, competitive ELISA, fluorescence titration, calorimetric methods, mass spectrometry (MS), and chromatography methods, such as high-performance liquid chromatography (HPLC). It is also understood measurements performed using such analytical methods are associated with detection limits, such as instrument detection limit, method detection limits, and limit of quantification.

Results from the cohorts 1-3 of the MAD study (Example 3) presented herein indicate that a delivered dose of the lipocalin mutein, or variant or fragment thereof, of about 2 mg or less than about 2 mg may result in a reduction of FeNO as a result of local lung exposure, without a substantive portion of the inhaled lipocalin mutein entering the circulatory system or detectable systemic exposure.

Results from cohorts 1-4 of the MAD study (Example 4) presented herein indicate that a delivered dose of the lipocalin mutein, or variant or fragment thereof, of about 2 mg or less than about 2 mg may result in a reduction of FeNO as a result of local lung exposure, without a substantive portion of the inhaled lipocalin mutein entering the circulatory system or detectable systemic exposure.

Results from cohorts 1-5 of the MAD study (Example 4) presented herein indicate that a delivered dose of the lipocalin mutein, or variant or fragment thereof, of about 2 mg or less than about 2 mg but greater than 0.2 mg may result in a reduction of FeNO as a result of local lung exposure, without a substantive portion of the inhaled lipocalin mutein entering the circulatory system or detectable systemic exposure. A delivered dose of the lipocalin mutein, or variant or fragment thereof, of about 2 mg or less than about 2 mg but about 0.6 mg or greater than about 0.6 mg, may result in a reduction of FeNO as a result of local lung exposure, without a substantive portion of the inhaled lipocalin mutein entering the circulatory system or detectable systemic exposure.

As used herein “local exposure” means there are sufficient levels of the of the inhaled lipocalin mutein present in the lung to interact with the target in the lung. This may occur without detectable target engagement in the blood or measurable concentrations of the lipocalin mutein in the blood or serum. As inhaled dose levels increase, the level of lung target engagement may increase and this may also be associated with substantive inhibition of target engagement in the blood and measurable concentrations of the lipocalin mutein in the blood or serum. The term “local lung exposure” refers to the lung concentration of the inhaled lipocalin mutein that is responsible for its lung target engagement. The reduction of fractional nitric oxide concentration in exhaled breath (FeNO) may be used to determine whether sufficient “local exposure” is achieved. In some other cases, in particular if the subject is human, since direct measurement of the amount of the lipocalin mutein that remains in the lung is difficult, determination of “local exposure” or “local lung exposure” may be carried out indirectly by determining the amount of the lipocalin mutein that enters the circulatory system.

Phosphorylation of STAT6 in the CD3+ T cell population may be used as a marker for systemic exposure of the lipocalin mutein. Determination of STAT6 phosphorylation (pSTAT6) may be carried out by any suitable method known to a person skilled in the art. For example, following administration of the lipocalin mutein to the subject, whole blood may be collected from the subject, stimulated with IL-4 and pSTAT6 in the CD3+ T cell subpopulation assessed using fluorescence-activated cell sorting (FACS), as described in the Examples section. Inhibition of IL-4 stimulated STAT6 phosphorylation in CD3+ T cells following administration of the lipocalin mutein to the subject indicates systemic exposure of the lipocalin mutein. The percentage inhibition of IL-4 stimulated STAT6 phosphorylation in CD3+ T cells by the lipocalin mutein may, for example, be determined relative to a control subject who has not been administered any lipocalin mutein. This may be the same subject (with IL-4 stimulated STAT6 phosphorylation in CD3+ T cells being assessed prior to administration of a lipocalin mutein) or in a different subject who has not been administered any lipocalin mutein.

Inhibition of IL-4 stimulated STAT6 phosphorylation in CD3+ T cells may be assessed by determining the IC₅₀ value, which is the half maximal inhibitory concentration of the lipocalin mutein; i.e. the concentration of the lipocalin mutein as measured in the plasma required to inhibit IL-4 stimulated STAT6 phosphorylation in 50% of CD3+ T cells. The IC₅₀ of the lipocalin mutein can be determined by constructing a dose-response curve and examining the effect of different concentrations of the lipocalin mutein on reversing IL-4 stimulated STAT6 phosphorylation in CD3+ T cells. IC₅₀ values can be calculated by determining the concentration of lipocalin mutein needed to inhibit STAT6 phosphorylation in half of the CD3+ T cells after stimulation with IL-4. Non-detectable or no significant inhibition of IL-4 stimulated STAT6 phosphorylation in CD3+ T cells may mean that there is no substantive portion of the inhaled lipocalin mutein entering the circulatory system or detectable systemic exposure.

Fractional nitric oxide concentration in exhaled breath (FeNO) may be used as a marker to determine the effectiveness of the lipocalin mutein in treating asthma. The person skilled in the art would readily be able to measure FeNO using known techniques, for example a FeNO test is done by the patients breathing out slowly and steadily into the mouthpiece attached to a hand-held monitor. The reading shows up on the monitor, with the result of the FeNO test showing how inflamed the airways are. A commonly used FeNO test is the American Thoracic Society (ATS) 2005 test.

The percentage reduction of FeNO by the lipocalin mutein may, for example, be determined relative to a control subject who has not been administered any lipocalin mutein. This may be the same subject (with FeNO being assessed prior to administration of a lipocalin mutein) or in a different subject who has not been administered any lipocalin mutein. A placebo may have been administered to this different subject.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a lipocalin mutein” includes one or more lipocalin muteins.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. The term, however, also includes the concrete number, e.g., “about 20” includes 20.

The term ‘at least about’ as used herein includes the concrete number e.g. ‘at least about 6’ includes 6.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

### EXAMPLES

### Example 1. A Human In Vitro Whole Blood Assay as an Evaluation of Human Immune Responses to PRS-060/AZD1402

To characterize the effect of PRS-060/AZD1402 on IL-4Rα signalling, human whole blood from healthy subjects was stimulated with IL-4 in the presence or absence of PRS-060/AZD1402 and quantified for the phosphorylation of signalling components and released soluble biomarkers.

Human whole blood was drawn from healthy volunteers and collected in a sterile tube containing heparin. Heparin-treated whole blood was stimulated with 8 ng/mL IL-4 for 15 minutes with increasing concentrations of PRS-060/AZD1402 or a reference IL4-Rα antibody, and phosphorylated STAT6 (pSTAT6) in the CD3+ T cell subpopulation was then assessed using fluorescence-activated cell sorting (FACS).

Additionally, heparin treated whole blood was stimulated with 8 ng/mL IL-4 for 24 hours with increasing concentrations of PRS-060/AZD1402 or a reference IL4-Rα antibody, followed by measurements of eotaxin-3, thymus- and activation-regulated chemokine (TARC), and macrophage-derived chemokine (MDC) using an enzyme-linked immunosorbent assay (ELISA).

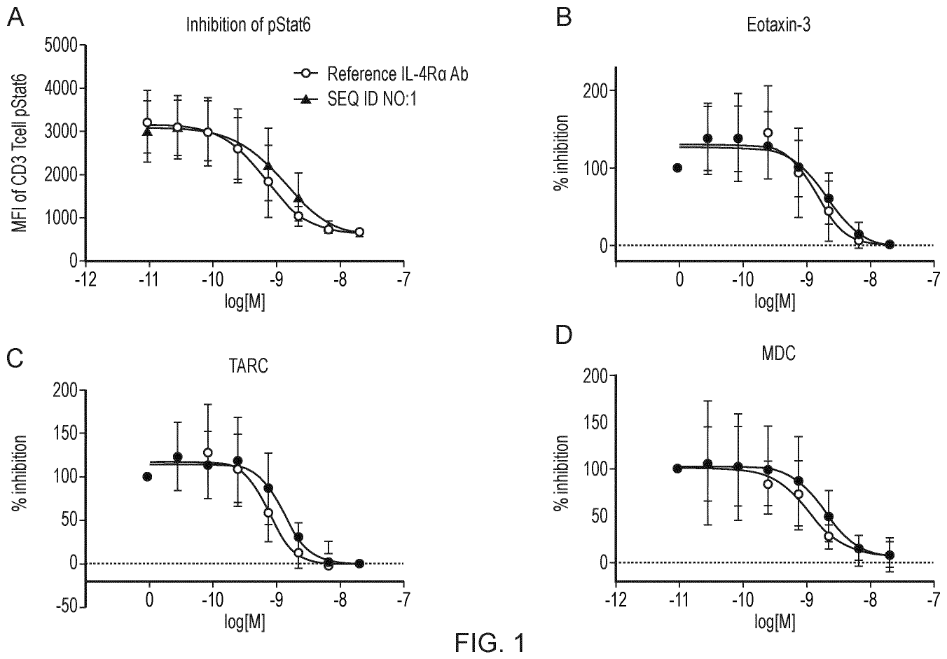

The results of representative experiments are depicted in FIG. 1 and fitted IC₅₀ values for PRS-060/AZD1402 and the reference IL4-Rα antibody inhibition of pSTAT6 and the release of soluble cytokines are summarized in Table 1. Stimulation of human whole blood with IL-4 resulted in increased levels of pSTAT6 and in the release of eotaxin-3, TARC, and MDC. PRS-060/AZD1402 inhibits pSTAT6 in a concentration-dependent manner and with similar potency to the reference IL-4Rα antibody. Inhibition of the release of the soluble cytokines eotaxin-3, TARC, and MDC by PRS-060/AZD1402, at equivalent potencies to the reference IL-4Rα antibody, was also observed.

The data suggest PRS-060/AZD1402 is capable of inhibiting IL-4Rα signalling in human whole blood and with IC₅₀ values comparable to those of the reference IL4Rα antibody. Furthermore, the low level of variation observed render this method suitable to detect the presence of systemic levels of PRS-060/AZD1402 following inhaled dosing. For example, pSTAT6 responses as well as downstream cytokine release in whole blood may be used in clinical trials to assess systemic exposure.

TABLE 1
Inhibition of pSTAT6 and the release of soluble cytokines

| | pSTAT | Eotaxin-3 | TARC | MDC |
|---|---|---|---|---|
| | IC₅₀ (nM) | IC₅₀ (nM) | IC₅₀ (nM) | IC₅₀ (nM) |
| PRS- | 1.3 | 2.1 | 1.3 | 2.0 |
| 060/AZD1402 | | | | |
| Reference IL- | 0.8 | 1.5 | 0.8 | 1.1 |
| 4Rα antibody | | | | |

Binding of IL-4 to its receptor (IL-4R) results in tyrosine phosphorylation of Janus kinase (Jak)3-1 and Jak-3, which further leads to the tyrosine phosphorylation of the IL-4Rα chain. After binding to the phosphotyrosine docking site on IL-4R through the Src homology 2 domain, Stat6 is phosphorylated by Jak kinases. Phosphorylated STAT6 (pSTAT6), released from IL-4R, forms a homodimer and translocates to the nucleus where it binds to a specific DNA sequence and triggers the transcription of its target genes (Nelms et al., Annu Rev Immunol, 1999, 17:701-738). The percent inhibition of pSTAT6 can be used as a direct measure reflecting the inhibition of the IL-4Rα following PRS-060/AZD1402 addition/administration.

Translocation of the pSTAT6 to the nucleus regulates a number of genes at the transcriptional level that are associated with type 2 immunity (Chen et al., 2003. J Immunol, 171:3627-3635). Induction of TARC/CCL17, MCD/CCL22 and Eotaxin-3/CCL26 at the transcriptional level has been demonstrated following IL-4 stimulation (see, for example, Wirnsberger et al., Eur J Immunol., 2006, 36(7):1882-1891, Rahal et al., Int J Radiat Oncol Biol Phys, 2018, 100(4): 1034-1043 and Hoeck and Woisetsschläger, J Immunol, 2001, 167(6):3216-3222). Furthermore, stimulation of human whole blood of healthy donors with IL-4 for 24 hours results in potent induction and cytokine release of TARC/CCL17, MCD/CCL22 and Eotaxin-3/CCL26 that can be inhibited by IL-4Rα. Thereafter these cytokines can be readily detected in the cell free portion of the blood.

### Example 2. A Dose Escalating Single Blind Study to Assess the Safety, Tolerability and Pharmacokinetics of Single Dose of PRS-060/AZD1402 Administered by Oral Inhalation or Intravenous Infusion in Healthy Subjects

### A. Study Objectives and Overview

This example describes a randomized, placebo-controlled, single-blind, single-dose escalation study conducted with oral inhalation or intravenous (IV) administration of a single dose of either PRS-060/AZD1402 or placebo to enrolled subjects. The primary objective of the study was to evaluate the safety and tolerability of single inhaled and single IV doses of PRS-060/AZD1402 in healthy male and female subjects. The secondary objective of the study was to evaluate the pharmacokinetics of PRS-060/AZD1402 after single inhaled and single IV doses of PRS-060/AZD1402 in healthy male and female subjects. Exploratory objectives of the study include PRS-060/AZD1402 effect on pharmacodynamic biomarkers such as inhibition of ex-vivo whole blood activation of the IL-4/IL-13 pathways.

Enrolled subjects were randomly assigned to a dose cohort. Each cohort included 8 subjects in total, consisting of 6 subjects for PRS-060/AZD1402 and 2 subjects for placebo. The 2 sentinel subjects per cohort were randomized 1:1 to PRS-060/AZD1402 and placebo and were dosed at least 24 hours before the remaining subjects in the cohort. The remaining subjects per cohort (randomized 5:1 to PRS-060/AZD1402 or placebo) received study medication not more than 40 minutes apart.

Subjects enrolled in the first cohort received the lowest dose of PRS-060/AZD1402 (0.25 mg nominal or metered dose; equivalent to a 0.1 mg delivered dose). The actual doses for each cohort were decided following review of the predefined exposure limits set by pre-clinical toxicology studies. The actual doses for the oral inhalation cohorts are summarized in Table 2. For administration, an InnoSpire Go nebulizer (Philips) was used. PRS-060/AZD1402 was formulated with a target protein concentration of 10 mg/mL or 50 mg/mL in an aqueous solution of phosphate buffered saline (PBS) (1.06 mM KH₂PO₄, 2.96 mM Na₂HPO₄, 154 mM NaCl, pH 7.4) and provided with a minimal extractable volume of 5.2 mL.

TABLE 2
Oral Inhalation Doses of PRS-060/AZD1402
and Matching Placebo

| | Nominal Doses (Delivered |
|---|---|
| | Doses) of PRS-060/AZD1402 and |
| Cohort | Matching Placebo (mg) |
| 1 | 0.25 (0.10) |
| 2 | 1.25 (0.50) |
| 3 | 5.00 (2.00) |
| 4 | 20.0 (8.00) |
| 5 | 60 (24.0) |
| 6 | 180 (72.0) |
| 7 | 400 (160) |

After safety evaluation of all cohorts of subjects who received oral inhalation doses (Cohorts 1 to 7), an additional 2 cohorts of subjects (who did not participate in the inhaled dose cohorts) were admitted for IV dosing. The 10 mg/mL PRS-060/AZD1402 was diluted in PBS for administration by infusion using a syringe pump. The additional 2 cohorts are summarized in Table 3.

TABLE 3
Intravenous Doses of PRS-060/AZD1402
and Matching Placebo

| | Intravenous Doses of PRS-060/AZD1402 |
|---|---|
| Cohort | and Matching Placebo (mg) |
| 8 | 1.0 |
| 9 | 2.0 |

Subjects were enrolled in the study based on the following criteria: (1) healthy male and female of non-childbearing potential (post-menopausal or surgically sterilized) subjects of 18 to 55 years of age; (2) body mass index (BMI) of 18-35 kg/m²; and (3) subjects who were non-smokers or ex-smokers who had not smoked in the last 6 months (determined by urine cotinine <500 ng/mL, at Screening visit). Subjects who met all the inclusion criteria were further screened for the following exclusion criteria: (1) history or clinical manifestations of any clinically significant medical disorder that, in the opinion of the investigator, might have put the subject at risk because of participation in the study, influence the results of the study or affect the subject's ability to participate in the study; (2) history of drug or alcohol abuse; (3) history of, or known significant infection including hepatitis A, B, or C, human immunodeficiency virus, tuberculosis (i.e., positive result for interferon-γ release assay, QuantiFERON TB-Gold), that might have put the subject at risk during participation in the study; (4) any clinically significant illness, infection, medical/surgical procedure, or trauma within 4 weeks of Day 1 or planned inpatient surgery or hospitalization during the study period; (5) any clinically significant abnormalities in clinical chemistry, haematology, or urinalysis results, as judged by the Principle Investigator; (6) subjects with any history of malignancy or neoplastic disease; (7) significant history of recurrent or ongoing ‘dry eye syndrome’ of any cause that might have been chronic or acute, that may affect the interpretation of safety data associated with the potential for anti-drug antibodies (ADAs) targeted to PRS-060/AZD1402 (structurally related to tear lipocalin); (8) subjects who had received live or attenuated vaccine in the 4 weeks prior to Day 1; (9) subjects with a disease history suggesting abnormal immune function; (10) history of anaphylaxis following any biologic therapy and known history of allergy or reaction to any component of the investigational product formulation; (11) inability to communicate well with the Investigator (i.e., language problem, poor mental development or impaired cerebral function); (12) participation in any clinical study for New Chemical Entity within the previous 16 weeks or a marketed drug clinical study within the previous 12 weeks or within 5 half-lives whichever was the longer before the first dose of study drug; (13) donation of 450 mL or more blood within the previous 12 weeks; (14) women who were pregnant; and (15) males who were sexually active with a female partner of childbearing potential and who had not had a vasectomy and who did not agree to double methods of contraception from Day 1 for 90 days.

### B. Study Procedures

Subjects were admitted to the study site in the afternoon of the day before Day 1 and remained in the study site until completion of the 48-hour measurements on Day 3. On the morning of Day 1, subjects received the treatment dose: a single inhaled dose or IV infusion of either active treatment (PRS-060/AZD1402) or placebo treatment. For subjects who received oral inhalation doses, study medication was provided at 10 mg/mL or 50 mg/mL in PBS and administered using an InnoSpire Go nebulizer (Philips). For subjects who received IV infusion, a 10 mL volume of PRS-060/AZD1402 in PBS was infused over a 60-minute period. Safety and PK assessments were performed at pre-determined time points during the study period. Subjects were discharged from the study site on Day 3 after all study assessments were completed and scheduled to return for Safety Follow-up, PK, and pharmacodynamic assessments on Day 7 (±1 day) and Day 30 (±3 days).

### C. Endpoints and Assessments

The primary endpoint of the study is safety/tolerability, assessed by adverse events (AEs), vital signs, forced expiratory volume 1 second (FEV₁), electrocardiogram (ECG), and laboratory safety tests on an ongoing basis during the study. An AE was defined as the development of an undesirable medical condition or the deterioration of a pre-existing medical condition following or during exposure to a pharmaceutical product, whether or not considered causally related to the product. Assessments of vital signs included body temperature, systolic and diastolic blood pressure readings (mm Hg), pulse (beats per minute (BPM)), and respiratory rate (breaths rate per minute (BRPM)). Blood and urine samples were collected for laboratory assessments, including haematology, serum chemistry, and urinalysis. Triplicate 12-lead ECGs were performed at pre-determined time points, prior to the blood collection, if collected at the same time.

Secondary endpoints of the study are PK parameters including: (1) serum (both oral inhalation and IV administration) maximum concentration (Cₘₐₓ), time to maximum concentration (Tₘₐₓ), terminal half-life (t_{1/2}), area under the curve from time zero to 24 hours post-dose (AUC₀₋₂₄), area under the curve from time zero to the last measurable concentration sampling time (Tₗₐₛₜ) (mass×time×volume-1) (AUCₗₐₛₜ), area under the curve from time zero to infinity (mass×time×volume−1) (AUC_{inf}), area under the curve from time zero to the last measurable concentration (AUCₗₐₛₜ), Cₘₐₓ/Dose, AUC₀₋₂₄ₕ/Dose, AUC₀₋ₗₐₛₜ/Dose, AUC_{inf/}Dose, and (mean residence time) MRT; (2) serum (IV administration only) volume of distribution at terminal phase (V_{z}), apparent volume of distribution at steady state (Vₛₛ), and systemic clearance (CL); (3) serum (oral inhalation only) apparent volume of distribution (V_{z}/F) and CL/F, and F_{inhalation/total} and mean absorption time (MAT) (both derived from IV PK data); and (4) urine (both oral inhalation and IV administration): total amount of drug excreted in urine (Ae), Ae(tₓ−tₓ₊₁), Ae(0−tₓ), fraction of dose excreted in urine (fe), fe(tₓ−tₓ₊₁), fe(0−tₓ), and renal clearance (CLᵣ).

Exploratory endpoints of the study include evaluating taste characteristics and PRS-060/AZD1402 effect on pharmacodynamic biomarkers such as inhibition of ex-vivo whole blood activation and exploratory systemic biomarkers relating to the IL-4/IL-13 pathways. Taste characteristics were evaluated using questionnaire. Plasma and serum were collected and used to assess potential biomarkers associated with the IL-4Rα pathway. Inhibition of ex-vivo whole blood activation was evaluated by stimulating whole blood collected from subjects with IL-4 (10 ng/ml human IL-4 for 15 minutes) and subsequently measuring phosphorylated STAT6 (pSTAT6) in CD3+ T cell subpopulations.

### D. Statistical Methods

For the primary endpoint, all subjects who provided informed consent and who received 1 dose of study drug were used for all analyses. Subjects were analyzed according to treatment received. Safety was assessed on the basis of AE reports, clinical laboratory data, vital signs, spirometry assessment, and 12-lead ECG parameters.

All AE summaries were restricted to treatment-emergent AEs (TEAEs) only, but all AEs were included in data listings. The TEAEs were defined as AEs that commenced on or after first dosing. Drug-related TEAEs were defined as TEAEs with possible, probable, or definite relationship to study drug. The number and percentage of subjects as well as number of events were presented for TEAE summaries. For summaries by MedDRA system organ class (SOC) and preferred term (PT), a subject was counted once at the SOC level and once at each PT within the SOC level. For summaries by SOC, PT, and severity, a subject was counted once at each severity level for which the event had occurred at the SOC level and at each severity level for which the event had occurred for each unique PT within that SOC level. Summaries by relationship to study drug were handled similar to the summaries by severity.

Laboratory data including haematology, serum chemistry, and urinalysis were obtained and summarized descriptively at each protocol scheduled visit—Screening, Day −1, Day 1, Day 2, Day 3, Safety Follow-up (Day 7±1), and 30-day Follow-up, by cohort and treatment, as absolute values and changes from baseline.

Spirometry assessment including FEV₁ (mL), forced expiratory volume 6 seconds (FEV₆) (mL), forced vital capacity (FVC) (mL), peak expiratory flow rate (PEFR) (L/min), and FEV₁/FVC ratio were obtained and summarized descriptively at each protocol scheduled time point (Screening; Pre-dose; 5 minutes, 40 minutes, 1 hour, 4 hours post-dose), by cohort and treatment, as absolute values and changes from baseline.

The 12-lead ECGs including RR interval (msec), PR interval (msec), QT interval (msec), QTcF interval (msec), and QTcB interval (msec) were obtained and summarized descriptively at each protocol scheduled timepoint (Screening; Pre-dose, 20 minutes, 30 minutes, 1 hour, 1.5 hours, 2 hours, 3 hours, 4 hours, 5 hours, 8 hours, 12 hours, 24 hours post-dose; Day 3; Safety Follow-up; 30-day Follow-up), by cohort and treatment, as absolute values and changes from baseline. Single 12-lead ECG was scheduled to be performed at 20 minutes, 30 minutes, and 1 hour post-dose on Day 1, and triplicate assessments at other time points. The mean of the triplicate ECG measurements performed pre-dose on Day 1 served as the subject's baseline-corrected QT (QTc) value for all post-dose comparisons.

For the secondary endpoint, all subjects who provided informed consent who received 1 dose of PRS-060/AZD1402 and had at least 1 evaluable blood sample for PK analysis collected were used for the analyses of PK parameter calculations, graphical displays of individual data, the listings of PK parameters data summation of PK concentration data and PK parameters and all other PK listings.

### E. Results

The results observed with all 72 enrolled subjects are summarized below. Of the 72 subjects, 54 subjects were randomized to receive PRS-060/AZD1402 and 18 subjects were randomized to receive placebo. The mean age of the participants was 26.4 years and the mean BMI was 24.5 kg/m². Eight subjects were allocated to each cohort. Within each cohort (Cohorts 1 to 9), 6 subjects received PRS-060/AZD1402 and 2 subjects received placebo. All 72 enrolled subjects received 1 dose of the study drug and completed this study. No subjects prematurely discontinued the study. The demographic and baseline characteristics are similar across groups and cohorts.

All 72 enrolled subjects who received 1 dose of the study drug were included in the Safety Population. A total of 37 subjects (51.4%) were included in the PK Population. Of the 37 subjects, 1 subject was in Cohort 3 and 36 subjects were in Cohorts 4 to 9 (6 subjects in each cohort). No subjects in Cohort 1 and Cohort 2 and no placebo subjects were included in the PK Population.

### (i) Primary Endpoint

Single inhaled doses and single IV doses of PRS-060/AZD1402 administered to healthy male subjects were well tolerated and safe.

A summary of TEAEs is provided in Tables 4 and 5 for all subjects and in Table 6 by cohort. Of the 72 subjects, the incidence of any TEAE was 34.7% (25 subjects): 33.3% in placebo (6 subjects) and 35.2% in PRS-060/AZD1402 (19 subjects). Subjects in all PRS-060/AZD1402 cohorts experienced at least 1 TEAE. The subjects in placebo Cohorts 1, 3, 4, and 8 experienced at least 1 TEAE. Of the 25 subjects who experienced any TEAEs, 10 subjects (40.0%) reported 11 events judged as possibly related to the study drug and 15 subjects (60.0%) reported 17 events judged as not related to the study drug. One placebo subject in Cohort 8 experienced headache judged as possibly related to study drug defined as a drug-related TEAE, but the event was mild in intensity and resolved without sequelae 1 hour after the event onset. None of the TEAEs were serious or led to discontinuation. No deaths occurred in this study. Placebo subjects and PRS-060/AZD1402 subjects both experienced the following TEAEs: headache, upper respiratory tract infection, and musculoskeletal chest pain. The most frequently reported TEAEs were headache experienced by 6 subjects (8%) and upper respiratory tract infection experienced by 5 subjects (7%). Other than headache and upper respiratory tract infection, no other events experienced by subjects were common to those receiving AZD1402/PRS-060 and placebo. Of the 25 subjects who experienced any TEAEs, 20 subjects (80.0%) reported mild TEAEs and 5 subjects (20.0%) reported moderate TEAEs. No subjects reported severe TEAEs.

TABLE 4
Overall Incidence of Treatment-Emergent Adverse
Events for All Subjects (Safety Population)

| | | PRS- | |
|---|---|---|---|
| | Placebo | 060/AZD1402 | Overall |
| | N = 18 | N = 54 | N = 72 |
| | n (%) m | n (%) m | n (%) m |
| TEAEs | 6 (33.3) 8 | 19 (35.2) 20 | 25 (34.7) 28 |
| Serious TEAEs | 0 | 0 | 0 |
| Drug-related TEAEs | 1 (5.6) 1 | 9 (16.7) 10 | 10 (13.9) 11 |
| TEAEs leading to study | 0 | 0 | 0 |
| discontinuation | | | |

TABLE 5
Incidence of TEA Es for All Subjects (Safety Population).

| | | AZD1402/ | |
|---|---|---|---|
| | Placebo | PRS-060 | Overall |
| System organ class | (n = 18) | (n = 54) | (n = 72) |
| Preferred term | n (%) m | n (%) m | n (%) m |
| Subjects with TEAEs | 6 (33) 8 | 19 (35) 20 | 25 (35) 28 |
| Nervous system disorders | 1 (6) 1 | 5 (9) 6 | 6 (8) 7 |
| Headache | 1 (6) 1 | 5 (9) 5 | 6 (8) 6 |
| Somnolence | 0 | 1 (2) 1 | 1 (1) 1 |
| Infections and infestations | 2 (11) 2 | 5 (9) 5 | 7 (10) 7 |
| URTI | 2 (11) 2 | 3 (6) 3 | 5 (7) 5 |
| Respiratory tract infection | 0 | 1 (2) 1 | 1 (1) 1 |
| Tonsillitis | 0 | 1 (2) 1 | 1 (1) 1 |
| Respiratory, thoracic and | 2 (11) 2 | 3 (6) 3 | 5 (7) 5 |
| mediastinal disorders | | | |
| Dry throat | 0 | 2 (4) 2 | 2 (3) 2 |
| Pleuritic pain | 0 | 1 (2) 1 | 1 (1) 1 |
| Throat irritation | 2 (11) 2 | 0 | 2 (3) 2 |
| General disorders and | 1 (6) 1 | 2 (4) 2 | 3 (4) 3 |
| administration site conditions | | | |
| Fatigue | 0 | 1 (2) 1 | 1 (1) 1 |
| Influenza-like illness | 0 | 1 (2) 1 | 1 (1) 1 |
| Application site dermatitis | 1 (6) 1 | 0 | 1 (1) 1 |
| Musculoskeletal and connective | 1 (6) 1 | 2 (4) 2 | 3 (4) 3 |
| tissue disorders | | | |
| Back pain | 0 | 1 (2) 1 | 1 (1) 1 |
| Musculoskeletal chest pain | 1 (6) 1 | 1 (2) 1 | 2 (3) 2 |
| Gastrointestinal disorders | 0 | 1 (2) 1 | 1 (1) 1 |
| Nausea | 0 | 1 (2) 1 | 1 (1) 1 |
| Investigations | 0 | 1 (2) 1 | 1 (1) 1 |
| Blood pressure increased | 0 | 1 (2) 1 | 1 (1) 1 |
| Injury, poisoning and procedural | 1 (6) 1 | 0 | 1 (1) 1 |
| complications | | | |
| Muscle injury | 1 (6) 1 | 0 | 1 (1) 1 |

TABLE 6
Incidence of Treatment-Emergent Adverse Events
by Cohort (Safety Population)

| | | | | |
|---|---|---|---|---|
| | | | PRS-060/ | |
| | | Placebo | AZD1402 | Overall |
| | | N = 2 | N = 6 | N = 8 |
| Cohort | Study Drug | n (%) m | n (%) m | n (%) m |

Any Treatment-Emergent Adverse Events

| | | | | |
|---|---|---|---|---|
| 1 | Oral | 1 (50.0) 2 | 1 (16.7) 1 | 2 (25.0) 3 |
| 2 | inhalation | 0 | 3 (50.0) 3 | 3 (37.5) 3 |
| 3 | | 2 (100) 2 | 3 (50.0) 3 | 5 (62.5) 5 |
| 4 | | 1 (50.0) 2 | 1 (16.7) 1 | 2 (25.0) 3 |
| 5 | | 0 | 1 (16.7) 1 | 1 (12.5) 1 |
| 6 | | 0 | 2 (33.3) 2 | 2 (25.0) 2 |
| 7 | | 0 | 3 (50.0) 3 | 3 (37.5) 3 |
| 8 | IV infusion | 2 (100) 2 | 3 (50.0) 4 | 5 (62.5) 6 |
| 9 | | 0 | 2 (33.3) 2 | 2 (25.0) 2 |

Drug-Related Treatment-Emergent Adverse Events

| | | | | |
|---|---|---|---|---|
| 1 | Oral | 1 (50.0) 2 | 1 (16.7) 1 | 2 (25.0) 3 |
| 2 | inhalation | 0 | 3 (50.0) 3 | 3 (37.5) 3 |
| 3 | | 2 (100) 2 | 3 (50.0) 3 | 5 (62.5) 5 |
| 4 | | 1 (50.0) 2 | 1 (16.7) 1 | 2 (25.0) 3 |
| 5 | | 0 | 1 (16.7) 1 | 1 (12.5) 1 |
| 6 | | 0 | 2 (33.3) 2 | 2 (25.0) 2 |
| 7 | | 0 | 3 (50.0) 3 | 3 (37.5) 3 |
| 8 | IV infusion | 2 (100) 2 | 3 (50.0) 4 | 5 (62.5) 6 |
| 9 | | 0 | 2 (33.3) 2 | 2 (25.0) 2 |

Abbreviations: AE = adverse event, m = number of events, N = number of subjects in the group, n = number of subjects in the specified category, TEAE = treatment-emergent adverse event.
Note:
TEAEs were defined as AEs that commenced on or after first dosing.
Note:
Percentages were based on the number of subjects in the Safety Population for each treatment group.

Clinical laboratory evaluation did not reveal clinically significant abnormalities or change from baseline. No individual clinically significant abnormalities were noted in this study. Similarly, no notable changes were observed in the vital signs, any of the pulmonary mechanic measurements, or ECG evaluation. The individual subject responses to the taste characteristic assessment were positive in that there was no significant taste or smell associated with study drug or placebo.

### (ii) Secondary Endpoint

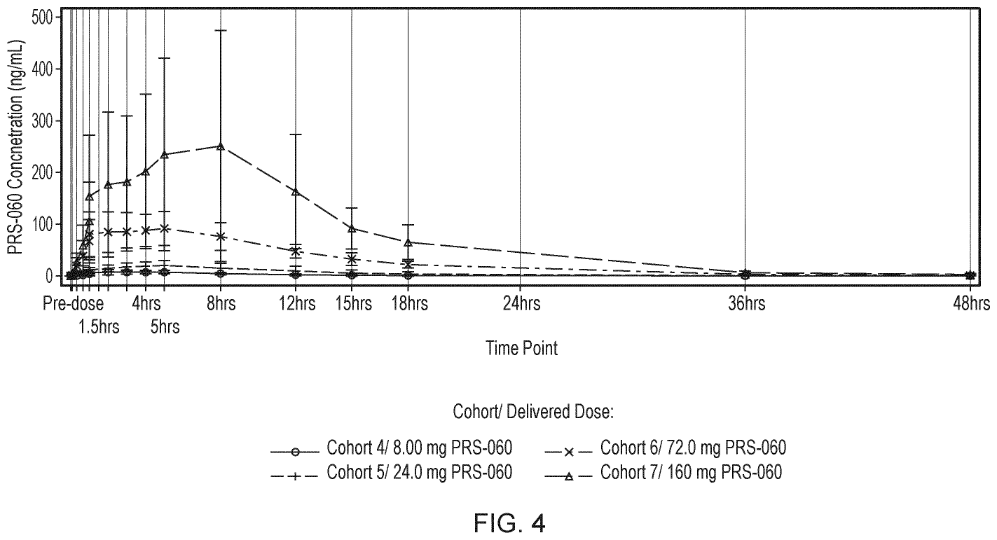

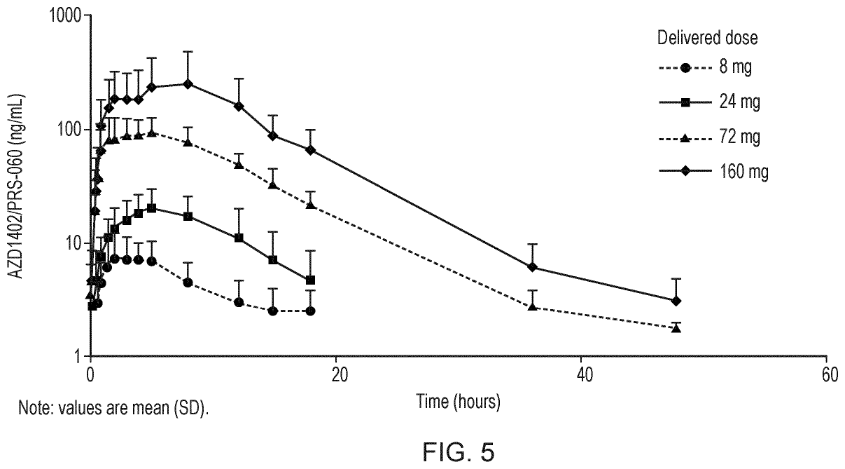

Following oral inhalation for Cohorts 1 to 7, serum PRS-060/AZD1402 PK profiles for all subjects in Cohort 1 (delivered dose 0.10 mg) and Cohort 2 (delivered dose 0.50 mg) were below the limit of quantitation (BLOQ) up until 30 days post-dose. For Cohort 3 (delivered dose 2.00 mg), 1 subject had detectable PRS-060/AZD1402 concentrations only at 4 hours post-dose (1.58 ng/ml) and at 5 hours post-dose (1.67 ng/mL), but were BLOQ for all other concentrations. Consequently, PK parameters were unable to be determined for the first 3 cohorts of this study. PK profiles of PRS-060/AZD1402 versus time were assessed from Cohort 4 (delivered dose 8.00 mg) onwards. A rank order increase in serum PRS-060/AZD1402 concentrations with increasing dose from Cohort 4>Cohort 5>Cohort 6>Cohort 7 was observed in mean PRS-060/AZD1402 concentration profiles (FIGS. 4 and 5). The corresponding serum PK parameters are summarized in Table 7. From the data, a greater than proportional increase in the PK parameters Cₘₐₓ and AUC with dose was observed. For example, a 2.2-fold increase in dose from Cohort 6 (72 mg delivered dose) to Cohort 7 (160 mg delivered dose) resulted in an approximate increase of 2.8-fold in Cₘₐₓ and AUC. A dose-proportional relationship was not observed, likely due to a high degree of intersubject variability at the highest inhaled delivered dose of Cohort 7 (67.2% and 87.1% for AUC_{inf}/Dose and Cₘₐₓ/Dose, respectively). The Tₘₐₓ occurred between 2 hours and 8 hours for all cohorts. In Cohorts 4 and 5, PRS-060/AZD1402 was detectable to around 18 hours post-dose, while the Tₗₐₛₜ was later in Cohorts 6 and 7. The terminal phase supported a mean (SD) t_{1/2} of 4.163 (1.7032) hours (Cohort 4), 4.100 (0.8974) hours (Cohort 5), 6.156 (0.7305) hours (Cohort 6) and 5.998 (0.6803) hours (Cohort 7).

TABLE 7
Serum PK Parameters Following PRS-060/AZD1402 Oral Inhalation at Delivered
Dose for Cohorts 4 to 7 (PK Population)

| | Cohort 4 | Cohort 5 | Cohort 6 | Cohort 7 |
|---|---|---|---|---|
| | 8.00 mg | 24.0 mg | 72.0 mg | 160 mg |
| Parameter | n = 6 | n = 6 | n = 6 | n = 6 |
| AUC₀₋₂₄ (h · ng/mL) | 84.65 (26.562)^{a} | 250.18 (112.847)^{b} | 1152.02 (377.432) | 3187.08 (2176.968) |
| AUC₀₋ₗₐₛt (h · ng/mL) | 61.94 (37.992) | 201.59 (118.163) | 1234.81 (400.317) | 3419.70 (2302.299) |
| AUC_{inf} (h · ng/mL) | 87.17 (27.753)^{a} | 261.50 (125.628)^{b} | 1252.14 (398.874) | 3445.99 (2314.929) |
| AUC₀₋₂₄/D (h · ng/mL/mg) | 10.58 (3.320)^{a} | 10.42 (4.702)^{b} | 16.00 (5.242) | 19.92 (13.606) |
| AUC₀₋ₗₐₛₜ/D (h · ng/mL/mg) | 7.74 (4.749) | 8.40 (4.923) | 17.15 (5.560) | 21.37 (14.389) |
| AUC_{inf}/D (h · ng/mL/mg) | 10.90 (2.969)^{a} | 10.90 (5.235)^{b} | 17.39 (5.540) | 21.54 (14.468) |
| Cₘₐₓ(ng/mL) | 8.278 (4.8164) | 21.155 (9.7602) | 93.017 (33.7836) | 266.833 (232.4749) |
| Cₘₐₓ/D (ng/mL/mg) | 1.035 (0.6020) | 0.881 (0.4067) | 1.292 (0.4692) | 1.668 (1.4530) |
| MRT_{inf} (h) | 7.76 (2.848)a | 8.90 (2.107)^{b} | 10.86 (1.616) | 11.49 (1.304) |
| Tₘₐₓ (h) (min, max) | 4.592 (2.10, 5.12) | 4.667 (4.12, 8.18) | 4.583 (1.65, 8.08) | 8.225 (1.73, 8.27) |
| t_{1/2} (h) | 4.163 (1.7032)^{a} | 4.100 (0.8974)^{b} | 6.156 (0.7305) | 5.998 (0.6803) |
| CL/F (L/h) | 95.314 (25.9729)^{a} | 104.141 (32.9355)^{b} | 64.293 (26.1382) | 64.573 (35.5707) |
| V_{z}/F (L) | 604.392 (390.2042)^{a} | 609.505 (248.1413)^{b} | 547.153 (259.6759) | 538.533 (253.7528) |

Abbreviations: D = dose, h = hour, min = minimum, max = maximum, MRT = mean residence time, PK = pharmacokinetics.
^{a}n = 2
^{b}n = 5
Note:
The values indicated for Tₘₐₓ are median (min, max)

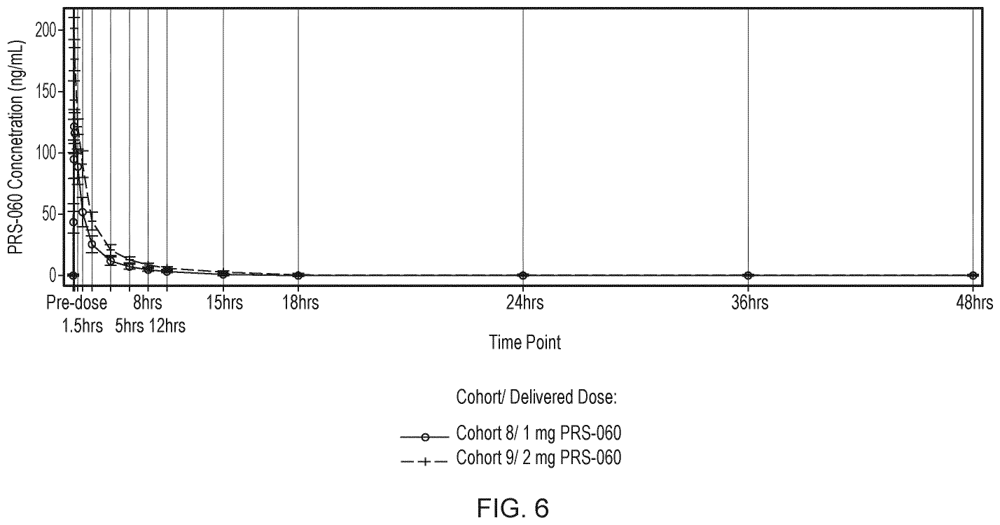

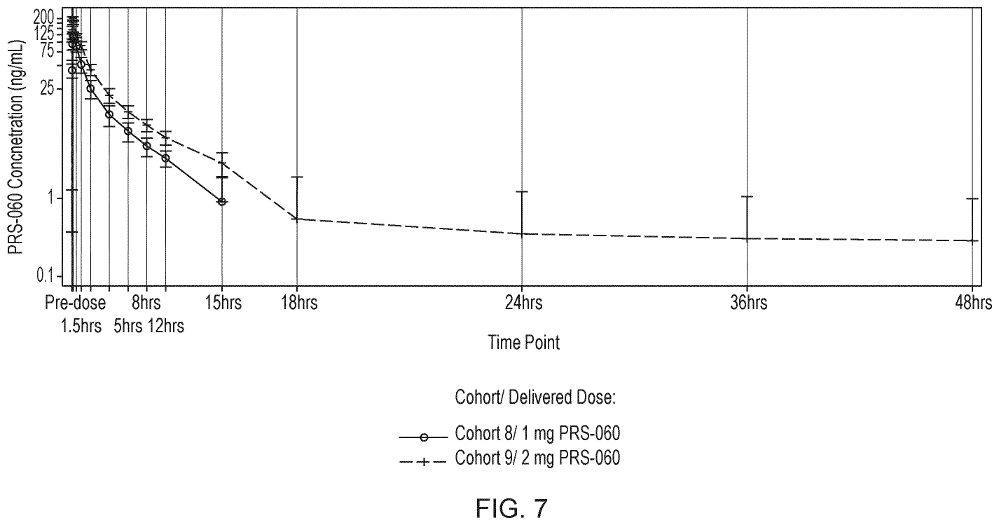

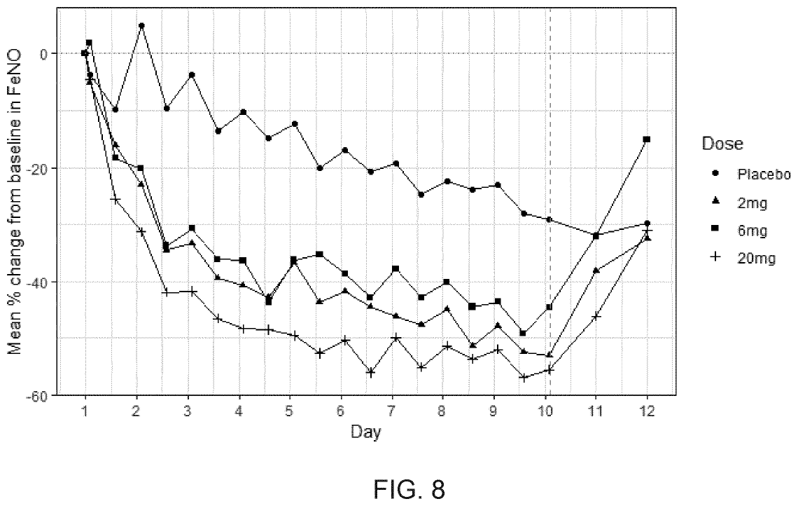

Following IV administration to Cohorts 8 and 9, mean (SD) serum PRS-060/AZD1402 indicated a rapid elimination phase with t_{1/2} of approximately half that observed in the inhaled doses in Cohorts 4 to 7 (FIGS. 6 & 7 and Table 8). For the 2-fold increase in IV dose (from 1 mg to 2 mg) between Cohorts 8 and 9, mean t_{1/2}, MRT_{inf}, V_{z}, Vₛₛ, and CL were similar while mean Cₘₐₓ, AUCₗₐₛₜ and AUC₀₋₂₄ increased by approximately 2-fold (Table 8). Of the dosed subjects, while PRS-060/AZD1402 levels were BLOQ on Days 7 and 30 post-dose in Cohort 8, for Cohort 9, 1 subject had PRS-060/AZD1402 levels up to 30 days post-dose. This subject was included in FIGS. 6 & 7, but was considered an outlier for the determination of terminal phase PK parameters (t_{1/2}, AUC_{inf}, CL/F and V_{z}/F) and not included in Table 8. Additionally, a Day 1 pre-dose level of 2.23 ng/mL was observed in the same Cohort 9 subject, which may reflect assay interference by human tear lipocalin presented in the serum. However, it was not expected to impact PK as this constituted 1.2% of Cₘₐₓ.

TABLE 8
Serum PK Parameters Following Intravenous Administration
for Cohorts 8 and 9 (PK Population)

| | Cohort 8 | Cohort 9 |
|---|---|---|
| | 1.0 mg | 2.0 mg |
| Parameter | n = 6 | n = 5 |
| AUC₀₋₂₄ (h · ng/mL) | 187.14 (32.385) | 316.47 (23.993) |
| AUC₀₋ₗₐₛₜ (h · ng/mL) | 180.27 (32.236) | 303.50 (22.321) |
| AUC_{inf} (h · ng/mL) | 187.28 (32.497) | 311.60 (23.099) |
| AUC₀₋₂₄/D | 187.14 (32.385) | 158.24 (11.997) |
| (h · ng/mL/mg) | | |
| AUC₀₋ₗₐₛₜ/D | 180.27 (32.236) | 151.75 (11.161) |
| (h · ng/mL/mg) | | |
| AUC_{inf}/D (h · ng/mL/mg) | 187.28 (32.497) | 155.80 (11.549) |
| Cₘₐₓ (ng/mL) | 123.333 (13.0639) | 201.500 (9.0277) |
| Cₘₐₓ/D (ng/mL/mg) | 123.33 (13.0639) | 100.750 (4.5139) |
| MRT_{inf} (h) | 1.42 (0.247) | 1.50 (0.120) |
| Tₘₐₓ (h) (min, max) | 0.983 (0.97,1.08) | 0.967 (0.97,1.00) |
| t_{1/2}(h) | 2.227 (0.7503) | 2.307 (0.1121) |
| CL (L/h) | 5.478 (0.9626) | 6.446 (0.4580) |
| Vₛₛ (L) | 7.637 (0.6943) | 9.661 (0.6971) |
| V_{z} (L) | 17.032 (3.9625) | 21.496 (2.4394) |

Abbreviations: D = dose, h = hour, min = minimum, max = maximum, PK = pharmacokinetics.
Note:
The values indicated for Tₘₐₓ are median (min, max).

As an absorption time was indicated by the longer t_{1/2} following single dose oral inhalation than after IV infusion, the mean absorption time (MAT) was determined based on the mean pooled MRT_{inf} in 11 subjects from both Cohort 8 at 1 mg infusion (6 Subjects) and Cohort 9 at 2 mg infusion (5 subjects) (Table 9). The mean (SD) MRT_{inf} across both IV cohorts was 1.45 (0.202) hours and was between 7.76 to 11.49 hours following inhalation of PRS-060/AZD1402 dose. Hence, the MAT was in the range of 7.45 to 10.04 hours when considering the higher dosed cohorts with the most complete data (where n=5 or 6).

Furthermore, the absolute bioavailability of the inhaled doses was determined by comparing mean AUC_{inf} following inhalation for Cohorts 4 to 7 with mean AUC_{inf} for IV Cohort 9 (2 mg) to range from approximately 6.99% to 13.8% (Table 10).

TABLE 9
Mean Absorption Time Determination

| Delivered | | Mean MRT_{inf} | Pooled Mean | |
|---|---|---|---|---|
| Dose (mg) | Cohort | (Inhalation) (h) | MRT_{inf} (IV) (h) | MAT**(h) |
| 8 | 4 (n = 2) | 7.76 | 1.45 | 6.31 |
| 24 | 5 (n = 5) | 8.90 | 1.45 | 7.45 |
| 72 | 6 (n = 6) | 10.86 | 1.45 | 9.41 |
| 160 | 7 (n = 6) | 11.49 | 1.45 | 10.04 |

Abbreviations: h = hour, IV = intravenous, MAT = mean absorption time, MRT = mean residence time.
Note:
**MAT = MRT_{inf} INH- MRT_{inf} IV

TABLE 10
Bioavailability Determination Cohort 9

| | | Mean AUC_{inf} | Mean | | Absolute |
|---|---|---|---|---|---|
| Delivered | | (Inhalation) | AUC_{inf} (IV) | Bioavail- | Bioavail- |
| Dose (mg) | Cohort | (h · ng/mL) | (h · ng/mL) | ability | ability (%) |
| 8 | 4 (n = 2) | 87.17 | 311.60 | 0.0699 | 6.99 |
| 24 | 5 (n = 5) | 261.50 | 311.60 | 0.0699 | 6.99 |
| 72 | 6 (n = 6) | 1252.14 | 311.60 | 0.1116 | 11.2 |
| 160 | 7 (n = 6) | 3445.99 | 311.60 | 0.1382 | 13.8 |

Abbreviation: AUC_{inf} = area under the curve from time zero to infinity, h = hour, IV = intravenous.

Urine PK profiles of PRS-060/AZD1402 were also evaluated. Urine samples were collected for inhaled dose to 48 hours post-dose. PRS-060/AZD1402 concentrations were detected in 3 subjects in this study. No urinary PRS-060/AZD1402 levels were observed in the IV cohorts (Cohorts 8 and 9). The summary of urinary PK parameters is shown in Table 11, which indicates a very low fraction of dose excreted as unchanged PRS-060/AZD1402 in the urine. Thus, urinary excretion of unchanged PRS-060/AZD1402 may be considered a minor elimination pathway.

The ADA results to 30 days post-dose for all cohorts were confirmed as negative.

TABLE 11
Urine Concentrations of PRS-060/AZD1402

| Cohort | | Collection | | Fraction of Dose |
|---|---|---|---|---|
| (dose*mg) | Subject | Interval | Ae (ng) | Excreted in Urine (%) |
| 6 (72.0 mg) | 002126 | 42-48 | 486 | 0.0007 |
| 7 (160 mg) | 002130 | 8-12 | 756.82 | |
| | | 12-18 | 1278.80 | 0.0013 |
| | 002144 | 8-12 | 2032.18 | |
| | | 42-48 | 1667.93 | 0.0023 |

Abbreviation: Ae = total amount of drug excreted in urine.

(iii) Exploratory Endpoint (pSTAT6 Inhibition):

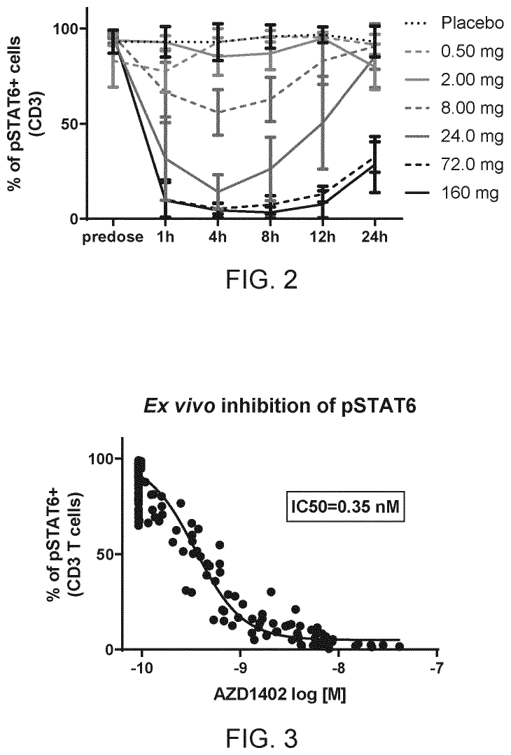

Ex-vivo whole blood stimulation with IL-4 was performed with subjects in Cohorts 2 to 7, and the corresponding pSTAT6 levels were determined. The mean and standard deviation of % pSTAT6+ CD3 cells in the subjects during the time-course of the sampling are presented in FIG. 2. Inhibition of pSTAT6 was observed from Cohort 4 (delivered dose 8.00 mg) onwards. The results from the subjects in Cohorts 4 and 5 (delivered doses 8.00 mg and 24.0 mg, respectively) demonstrated the highest inhibition of the % of the pSTAT6+ CD3 cells between 4 to 8 hours post inhalation. The results from the subjects in cohorts 6 and 7 (delivered doses 72.0 mg and 160 mg, respectively) demonstrated a potent and prolonged inhibition of the % of the pSTAT6+ CD3 cells from 1 hour up to 24 hours post-dose.

PK/PD analysis of the inhibition of ex vivo whole blood activation (FIG. 3) demonstrate a dose-dependent inhibition of the downstream STAT6 phosphorylation, with low variation between subjects, following inhalation of PRS-060/AZD1402. The IC₅₀ value was calculated at 0.35 nM.

### F. Discussion and Conclusions

Systemic exposure of inhaled PRS-060/AZD1402 was observed at delivered dose of 8.00 mg or higher. The slow decline in serum PK following inhalation indicates absorption-driven elimination. A high degree of variability on serum PRS-060/AZD1402 levels at the highest inhaled delivered dose (160 mg) prevented a dose-proportional relationship being defined. For the 2-fold increase in IV doses (1 mg to 2 mg), mean t_{1/2}, MRT_{inf}, V_{z}, Vₛₛ, and CL were similar, while mean Cₘₐₓ, AUCₗₐₛₜ, and AUC₀₋₂₄ increased by approximately 2-fold.

A protein with the molecular weight (17 kDa) of PRS-060/AZD1402 could be cleared renally and have a low tissue distribution. A Vₛₛ value of approximately 10 L determined in the IV PK cohorts, confirmed the low tissue distributions. Urinary PK parameters were not confirmed because urinary excretion of unchanged PRS-060/AZD1402 was not detectable in most subjects' urine and otherwise at very low levels. This indicated that urinary excretion was a minor elimination pathway, at least for unchanged PRS-060/AZD1402.

Inhibition of pSTAT6 in CD3+ cells present in the blood was correlating with systemic exposure and observed from 8.00 mg delivered dose onwards. The variability in the % of the pSTAT6+ CD3 cells in the subjects for each cohort was due to the variation of PRS-060/AZD1402 systemic exposure. These results indicate PRS-060/AZD1402 inhalation does not affect the stability and activity of the molecule, which reaches the systemic circulation and can potently inhibit signalling downstream of the IL-4Rα.

No positive ADA results that indicated a potential risk with the use of PRS-060/AZD1402 were recorded for any subjects from all cohorts for oral inhalations and IV administrations. Further, no ADAs were detected in any subjects.

Overall, no safety concerns were observed in this study. The incidence of any TEAE was 34.7% (25 subjects): 33.3% in placebo (6 subjects) and 35.2% in PRS-060/AZD1402 (19 subjects). The incidence seen in the PRS-060/AZD1402 subjects was similar to that seen in the placebo subjects. The incident rate of any TEAEs was independent of the dosage administered. The most frequently reported TEAE was headache in 6 subjects (8.3%) with 7 events followed by upper respiratory tract infection in 5 subjects (6.9%) with 5 events.

None of the TEAEs were reported as definitely related, probably related, or definitely not related. The incidence of drug-related TEAEs reported as possibly related was 13.9% (10 subjects [9 subjects in PRS-060/AZD1402 and 1 subject in placebo]). The drug-related TEAEs included headache, somnolence, dry throat, pleuritic pain, nausea, respiratory tract infection, and musculoskeletal chest pain.

The majority of the TEAEs were mild, and all events were reversible. No severe TEAEs were reported. None of the TEAEs were serious or led to discontinuation. No deaths occurred in this study.

None of the clinical laboratory evaluations resulted in any clinically significant abnormalities or change from baseline. No individual clinically significant abnormalities were noted in this study.

No notable changes were observed in the vital signs, any of the pulmonary function measurements, including the FEV₁/FVC ratio, or ECG evaluation.

The individual subject responses to the taste characteristic assessment indicated that there was no unpleasant taste or smell associated with PRS-060/AZD1402 or placebo. Based on this, repeated use of the study drug (nebulized drug product) was considered possible.

In conclusion, single inhaled doses and single IV doses of PRS-060/AZD1402 in healthy male adult subjects were safe and well tolerated. A dose related systemic exposure of PRS-060/AZD1402 after inhalation was observed with a profile indicating absorption-driven elimination and correlated closely with the PD effects of the molecule. Doses may also be selected at which no systemic exposure of inhaled PRS-060/AZD1402 is observed.

### Example 3. A Dose-Escalating, Single-Blind Study to Assess the Safety, Tolerability, and Pharmacokinetics of Multiple Doses of PRS-060/AZD1402 Administered by Oral Inhalation in Subjects with Mild Asthma

Example 3 provides data from this study for cohorts 1-3, with data for cohorts 1-5 being provided in Example 4. As the clinical trial has not yet completed, the data lock for the overall clinical study, and final data outputs have not yet been produced for the study report.

### A. Study Objectives and Overview

This example describes a placebo-controlled, single-blind, randomized, dose-escalating study conducted with oral inhalation of multiple doses of PRS-060/AZD1402 to enrolled subjects with mild asthma. The primary objective of the study was to evaluate the safety and tolerability of multiple inhaled doses of PRS-060/AZD1402 in male and non-pregnant, non-breastfeeding female subjects with mild asthma. The secondary objectives of the study were to evaluate the serum and urine pharmacokinetics (PK) of PRS-060/AZD1402 after multiple inhaled doses of PRS-060/AZD1402 in mild asthmatic male subjects and mild asthmatic non-pregnant, non-breastfeeding female subjects, to evaluate the potential development of anti-drug antibodies (ADAs) against PRS-060/AZD1402, and to evaluate the change from baseline in fractional nitric oxide concentration in exhaled breath (FeNO) in mild asthmatics receiving multiple inhaled doses of PRS-060/AZD1402 or placebo. Exploratory objectives of the study include PRS-060/AZD1402 effect on pharmacodynamic biomarkers such as inhibition of ex-vivo whole blood activation of the IL-4/IL-13 pathways.

53 subjects who satisfied the inclusion and exclusion criteria (see below) were enrolled and allocated into 5 cohorts: Cohort 1 and 2 with 8 subjects (both including 6 active, 2 placebo subjects each), Cohort 3 with 18 subjects (12 active, 6 placebo), Cohort 4 with 8 subjects (6 active and 2 placebo subjects) and Cohort 5 with 11 subjects (9 active, 2 placebo). There were 2 sentinel subjects dosed for each cohort randomized 1:1 to active PRS-060/AZD1402 and placebo except for cohort 5. The remaining subjects per cohort were randomized 5:1 for Cohorts 1 and 2, 11:5 for Cohort 3 and 5:1 for Cohort 4 to PRS-060/AZD1402 or placebo.

Enrolled subjects received multiple doses of PRS-060/AZD1402 or matching placebo as a nebulized solution via oral inhalation, twice daily (BID) (every 12 hours, for 9 days) from Day 1 to Day 9 and once in the morning on Day 10 for cohorts 1 to 5. Subjects enrolled in cohort 1 received PRS-060/AZD1402 at 5.0 mg nominal dose; equivalent to a 2.0 mg delivered dose given twice daily except for on day 10 when only the first morning dose was administered. Subjects enrolled in Cohort 2, 3, 4 and 5 received doses per Table 12. The doses were decided following review of the data obtained in the Phase 1 Single ascending dose study, as described in Example 2, and predefined exposure limits set by pre-clinical toxicology studies. There was a minimum of 7 days between completion of dosing of the preceding cohort and initiation of dosing for the next cohort, except for cohort 5. For each cohort, sentinel subjects were dosed at least 48 hours before the remaining subjects in the cohort, except for cohort 5. Following a review of the sentinel subjects' data to confirm that the results were favorable, the same dose level was administered to the remaining eligible subjects for the cohort. The remaining subjects were dosed at least 30 minutes apart from start of inhalation. All safety data and PK data were reviewed to determine whether to continue to the next cohort or to delay, stop, or modify downwards dose escalation.

TABLE 12
Doses of PRS-060/AZD1402 and Matching Placebo

| | Multiple Nominal Doses (Delivered |
|---|---|
| | Doses) of PRS-060/AZD1402 |
| Cohort | and Matching Placebo (mg) |
| 1 | 5 (2.0) |
| 2 | 15 (6.0) |
| 3 | 50 (20) |
| 4 | 150 (60) |
| 5 | 0.5 (0.2) |

Subjects were enrolled in the study based on the following criteria: (1) body Mass Index (BMI) of 18 to 35; (2) subjects who were non-smokers or ex-smokers who had smoked no more than twice in the 3 months prior to screening (determined by urine cotinine <500 ng/mL, at Screening visit); (3) males and non-pregnant, non-breastfeeding females; (4) males who were sexually active with women of childbearing potential agree to follow a highly effective method of contraception for the duration of treatment with study drug as well as for an additional 90 days after last dose of study drug. Women of childbearing potential who were sexually active with a fertile man agree to follow instructions for double methods of contraception for the duration of their participation in the trial and for 90 days after the last dose of the study drug; (5) documented diagnosis of mild asthma; (6) 18 to 55 years of age; (7) lung function≥70% predicted for FEV₁ and FEV₁/Forced Vital Capacity (FVC) ratio≥0.7; (8) FeNO≥35 ppb at screening and during pre-qualification for the study.

Additionally, subjects who met any of the following criteria were not enrolled: (1) history or clinical manifestations of any clinically significant medical disorder that, in the opinion of the investigator, may have put the subject at risk because of participation in the study, influence the results of the study, or affected the subject's ability to participate in the study. A history of drug or alcohol abuse; (2) history of, or known significant infection, including hepatitis A, B, or C, Human immunodeficiency Virus (HIV), tuberculosis (i.e., positive result for interferon [IFN]-γ release assay [IGRA], QuantiFERON® TB-Gold), that may have put the subject at risk during participation in the study; (3) history of cancer within the last 10 years (20 years for breast cancer) except for basal and squamous cell carcinoma of the skin or in situ carcinoma of the cervix treated and considered cured. Any history of lymphoma was not allowed; (4) any clinically significant illness, infection, medical/surgical procedure, or trauma within 4 weeks of Day 1 or planned inpatient surgery or hospitalization during the study period; (5) any clinically significant abnormalities in clinical chemistry, hematology, or urinalysis results, as judged by the Principal Investigator; (6) significant history of recurrent ongoing ‘dry eye syndrome’ of any cause that may have been chronic or acute, that may have affected the interpretation of safety data associated with the potential for ADAs targeted to PRS-060/AZD1402 (structurally related to tear lipocalin); (7) subjects who had received live or attenuated vaccine in the 4 weeks prior to Day 1; (8) subjects with a disease history suggesting abnormal immune function; (9) history of anaphylaxis following any biologic therapy and known history of allergy or reaction to any component of the investigational product formulation; (10) inability to communicate well with the Investigator (i.e., language problem, poor mental development, or impaired cerebral function); (11) participation in any clinical study for a New Chemical Entity within the previous 16 weeks or a marketed drug clinical study within the previous 12 weeks or within 5 half-lives, whichever was the longer, before the first dose of study drug; (12) donation of 450 mL or more blood within the previous 12 weeks; (13) women who were pregnant or breastfeeding, or were planning on becoming pregnant within the study period or 90 days following the last dose of study drug; (14) males who were sexually active with a female partner of childbearing potential and who had not had a vasectomy and who did not agree to a highly effective method of contraception from Day 1 to 90 days post last dose of study drug. Females of childbearing potential who were sexually active with a fertile male partner and who did not agree to double methods of contraception with at least one barrier from Day 1 to 90 days post last dose of study drug; (15) life-threatening asthmatic episode in the past; (16) used any of the following medicines within the specified time before Screening: long-acting β2 agonists (none for 4 weeks prior to Screening), anti-IgE or anti-IL-5 therapy (for 6 months prior to Screening), inhaled corticosteroids (>500 μg per day of beclometasone dipropionate (BDP) or equivalent) within 16 weeks prior to Screening, any inhaled corticosteroids at screening or within 4 weeks prior to screening or at randomization, oral or injectable steroids for the treatment of asthma or respiratory tract infection within 5 years prior to Screening, intranasal steroids within 4 weeks prior to Screening, topical steroids within 4 weeks prior to Screening, leukotriene antagonists within 2 weeks prior to Screening, or xanthines (excluding caffeine), anticholinergics, or cromoglycate within 1 week prior to Screening.

### B. Study Procedures

The study comprised pre-study assessments during Screening (Day −21, 21 days prior to administration of study drug, to Day −2). FeNO was assessed at screening for eligibility and on Day −1 (Run-in) to confirm eligibility. Subjects with FeNO≥35 ppb on both occasions were randomized into the study to receive PRS-060/AZD1402 or placebo. On Day −1 (Run-in), one day before they received the first dose of PRS-060/AZD1402 or matching placebo, subjects checked into the hospital/study site; subjects checked out 48 hours (Day 12) after administration of the final dose (Day 10). On the morning of Day 1, study drug of either PRS-060/AZD1402 or placebo were administered using an InnoSpire Go nebulizer (Philips). Safety and PK assessments were made at pre-determined time points during the study period. A full PK profile was performed on Days 1 and 10 following the administration of the morning dose. The subjects were then discharged from the clinic on the morning of Day 12, and returned for safety follow-up, PK, and PD assessments on Day 17 (±1 day) and Day 40 (±3 days) after they received the last dose of study drug on Day 10.

### C. Endpoints and Assessments

The primary endpoint of the study is safety/tolerability, assessed by adverse events (AEs), vital signs, forced expiratory volume 1 second (FEV₁), electrocardiogram (ECG), and laboratory safety tests on an ongoing basis during the study. Subjects were monitored for AEs during study participation (beginning at the time study drug was first administered) and until 30 days after the last dose of study drug. Any ongoing serious AEs (SAEs) were followed until resolution or stabilization. Assessments of vital signs included body temperature, systolic and diastolic blood pressure readings (mm Hg), pulse (beats per minute [BPM]), and respiratory rate (breaths rate per minute [BRPM]). Blood and urine samples were collected for laboratory assessments, including hematology, serum chemistry, urinalysis, and pregnancy screen. Triplicate 12-lead ECGs were performed at pre-determined time points, prior to the blood collection if collected at the same time.

For the primary endpoint, all subjects who received 1 dose of PRS-060/AZD1402 were included in the safety analyses. Safety was assessed on the basis of AEs, vital signs, pulmonary function tests (PFTs), ECGs, and laboratory data. All AE, physical exam, vital signs, PFTs, and ECG assessments plus safety laboratory abnormalities of potential clinical concern were described. Safety data are presented in tabular and/or graphical format and summarized descriptively by dose cohort and time as appropriate. Absolute value data and changes from baseline data are summarized as appropriate.

AEs were coded using the Medical Dictionary for Regulatory Activities (MedDRA) System Organ Classes and Preferred terms. All AEs were characterized as pretreatment and treatment-emergent AEs (TEAEs) according to the onset date before or after the first dosing. Incidence tables of subjects with AEs are presented for all AEs by maximum severity, SAEs, AEs assessed as related to study drug, and AEs resulting in discontinuation of study drug.

For ECG analyses, triplicate ECG measurements were made for pre-dose and single measurements at 20, 30 and 60 minutes post dose and in triplicate for every measurement after. The mean of the triplicate ECG measurements performed pre-dose on Day 1 served as the patient's baseline-corrected QT interval (QTc) value for all post-dose comparisons. Changes in ECG and laboratory measurements are summarized.

Secondary endpoints of the study are PK parameters, occurrence of anti-drug antibodies (ADAs) against PRS-060/AZD1402 in serum, and changes from baseline of FeNO levels. Venous blood samples and urine samples for the PK analysis and ADA assessment were collected at pre-determined time points. The following PK parameters were determined: Cₘₐₓ, Cₐᵥₑ, Tₘₐₓ, area under the curve from time zero to 12 hours post-dose (AUC₀₋₁₂), AUC₀₋₂₄, AUC₀₋ₗₐₛₜ, AUC_{inf}, accumulation ratio for AUC from time zero to the end of the dosing period (Rac AUC_{0-τ}), Rac Cₘₐₓ, temporal change parameter (TCP), dose-normalized exposure parameters (AUC₀₋₂₄/Dose, AUC₀₋ₗₐₛₜ/Dose, AUC_{inf}/Dose), t_{1/2}, apparent clearance for inhaled administration (CL/F), and volume of distribution based on the terminal phase (V_{z}/F), Ae, fe of PRS-060/AZD1402 and CLᵣ. Ae and fe were accumulatively determined (Ae[tₓ−tₓ₊₁], Ae[0−tₓ], fe[tₓ−tₓ₊₁] and fe[0−tₓ]) for each urine collection interval. The change in FeNO levels from baseline compared to placebo was assessed as an index of pharmacological activity. Airway inflammation was evaluated using a standardized single-breath FeNO test, performed during Screening and any follow up visit and 5 times daily during the dosing period (once pre-dose, and twice after each BID [2 times daily] dose). The FeNO testing was completed in the same manner at every study visit. Subjects inhaled to total lung capacity through the NIOX VERO® Airway Inflammation Monitor and then exhaled for 10 seconds at 50 mL/sec (assisted by visual and auditory cues). The value obtained was recorded and the process repeated for a total of 2 measurements (with up to 2 repeats).

Exploratory endpoints of the study included PRS-060/AZD1402 effect on PD biomarkers, such as inhibition of ex-vivo whole blood activation and exploratory systemic biomarkers relating to the IL-4/IL-13 pathways, and soluble biomarker analysis from plasma and serum samples prior, during, and after the duration of the dosing. For exploratory analysis, plasma, serum, peripheral blood mononuclear cells (PBMCs) and whole blood were collected. Plasma and serum were used to assess potential soluble biomarkers associated with the IL-4Rα pathway. Ex-vivo stimulation of whole blood was used to assess the systemic target engagement. The inhibition of whole blood activation was evaluated by ex-vivo stimulating whole blood collected from subjects with IL-4 and subsequently measuring phosphorylated STAT6 (pSTAT6) in CD3+ T cell subpopulations following inhalation at pre-defined timepoints. DNA was used in an attempt to identify genotypes relating to the disease. mRNA analysis was performed in an attempt to identify patients with gene signatures that were associated with the IL-4Rα pathway and were the most likely to benefit by the intervention.

### D. Data Analysis

(i) PK

For the secondary endpoints, PK profiles of PRS-060/AZD1402 on Day 1 and Day 10 for the first 3 cohorts were investigated during the course of the study and the PK population was used in data analyses. The exposure PK parameters were derived according to standard noncompartmental analytical procedures. The software used was Phoenix™ WinNonlin® v 8.0 (Pharsight Corporation, USA). Descriptive statistics of PK exposure parameters included arithmetic mean, and standard deviation (SD), per Table 19 and descriptive mean serum concentration versus time profiles were generated.

(ii) Anti-Drug Antibody Formation

The immunogenicity of PRS-060/AZD1402 (anti-PRS-060/AZD1402 antibody formation) was investigated during the course of the study.

(iii) PD—FeNO

FeNO was defined as the PD marker for PRS-060/AZD1402. The available PD data for any subjects excluded from the PD analysis were listed and only subjects in the PD analysis set were included in the descriptive summary tables and summary/mean figures. Inferential statistics was performed on the PD analysis set to assess the change from baseline in FeNO for all dose groups individually and for the PRS-060/AZD1402 dose groups compared to placebo. FeNO is a log normally distributed endpoint which implies that the analysis was performed on the log-scale. In the presentation of results, estimated mean differences between active and placebo were transformed to linear scale and expressed as percentage reduction from baseline in active group relative to placebo group. At the completion of cohort 3, a data snapshot for the purposes of an interim analysis was performed (see section (v) below), to assess the variability of the FeNO measurement in order to assure proper powering of the cohorts, and to assess preliminary estimates of change from baseline for each of the first three doses relative to placebo.

(iv) Analysis of FeNO

Placebo subjects were pooled from all three cohorts into one group containing 10 patients. Cohorts 1 and 2 each included 6 patients on active treatment, and cohort 3 had 12 patients. Each patient contributed 20 FeNO measurements: baseline value, recordings 2 h post morning dose and 2 h post evening dose for Day 1 to Day 9, and 2 h post morning dose on Day 10.

Estimates of mean difference in log FeNO between each active group and placebo were derived from a non-linear mixed effect model, where the non-linear part was a sigmoidal emacs model of the form

$$\frac{A \cdot {t}^{h}}{{t}_{mid}^{h} + {t}^{h}}$$

The asymptote parameter A was modelled by a fixed treatment group effect and a subject specific random effect:

Aᵢₖ=βₖ+bᵢ

for treatment group k (k=placebo, 5 mg, 15 mg and 50 mg nominal dose) and subject i (i=1 through to 34). The random effect accounted for the within patient correlation and allowed for subject specific asymptotes. To allow for a different time-course effect in the placebo group, the t_{mid}-parameter included two fixed effect levels:

t_{mid}=β_{Active}+β_{Placebo}

For graphical visualization of the data the observed FeNO reductions were plotted over treatment period, (Days 1 to Day 10), and Days 11 and 12. FeNO measurement from Days 11 and 12 were included in graphs to illustrate the return of FeNO towards its baseline level.

### E. Results

(1) FeNO Results

FeNO baseline mean (SD) across all cohorts (n=34) was 75.9 (41.0) ppb, median was 62 ppb. The estimated percent reduction in placebo group after 10 days of treatment was 25.2%. Table 13 shows percent reduction in each of the dose groups relative to the placebo group.

TABLE 13
FeNO results: Mean percent reduction from baseline relative to placebo.
Estimated treatment effects represent the reduction at end of
treatment (Day 10).

| Delivered Dose of | | |
|---|---|---|
| PRS-060/AZD1402 | | |
| and Matching | Percent reduction vs placebo | |
| Placebo (mg) | (95% CI) | P-value* |
| 20 mg | 40.3%, (21%, 55%) | <0.001 |
| 6 mg | 24.8%, (−5.5%, 46%) | 0.099 |
| 2 mg | 30.9%, (3.0%, 51%) | 0.03 |

*Two-sided test of null hypothesis “no difference between active and placebo”

(2) PK Results

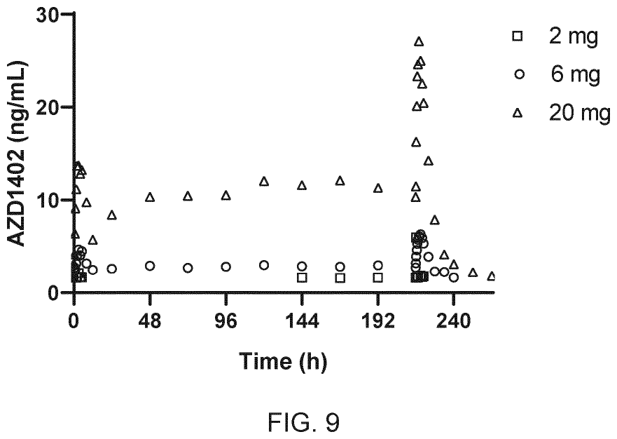

Limited serum exposure was observed after subjects received a delivered dose of 2 mg in Cohort 1, which was insufficient for PK parameter calculation. More complete exposure data was observed after the subjects received delivered doses of 6 mg in Cohort 2 and after 20 mg in Cohort 3 allowing PK parameters to be derived (FIG. 9). The main findings from cohorts 1-3 were as follows:
AUC and Cₘₐₓ exposures increased with dose (Table 14).
Pre-dose samples were taken throughout the 10 day dosing period and the observed serum levels indicated that steady state had been achieved by the end of the 2nd day of dosing (FIG. 9)
Exposures after dosing on Day 10 following 9 days twice-daily administration were higher than after the 1st dose on Day 1. The increase observed was reasonably consistent with the dosing interval of 12 h and the PK properties derived in the single ascending dose study (example 2).

No urinary PK analysis has been completed.

In Cohort 1, 2 subjects returned low positive ADA results on Day 17, subsequent responses on Day 40 were negative. In Cohort 2 there were no ADA findings.

TABLE 14
Analysis exposure data on Day 10 of dosing

| Delivered | | | | |
|---|---|---|---|---|
| Dose of | | | | |
| PRS-060/ | PK parameter mean value (SD) | | | |
| AZD1402 | Cₘₐₓ (ng/mL) | | AUC₍₀₋₁₂₎(h * ng/mL) | |
| (mg) | Day 1 | Day 10 | Day 1 | Day 10 |
| 2 | Not | Not | Not | Not |
| | determined* | determined* | determined* | determined* |
| 6 | 5.00 (3.39) | 6.33 (1.63) | 38.5 (21.4) | 54.0 (13.9) |
| 20 | 15.2 (7.39) | 27.8 (13.0) | 122 (65.0) | 210 (97.6) |

*Not determined due to lack of subjects with measurable exposures

(3) Exploratory Endpoint—Target Engagement by Monitoring pSTAT6 Inhibition in CD3+ Cells

### Results:

Inhibition of STAT6 phosphorylation was evaluated to assess PRS-060/AZD1402 target engagement.

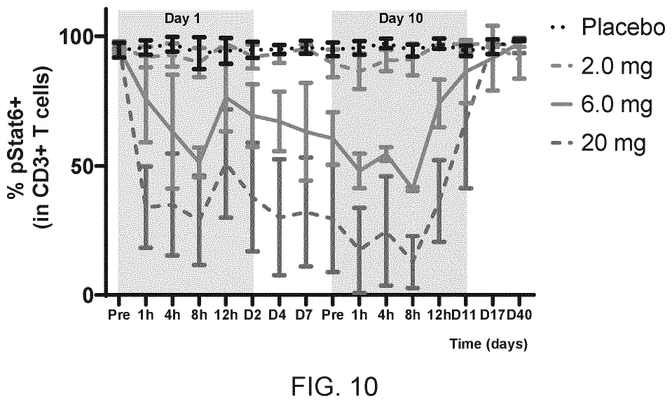

Ex-vivo whole blood stimulation with IL-4 was performed in blood of subjects enrolling in one of the sites from Cohorts 1 to 3, and the corresponding pSTAT6 levels were determined. Whole blood was collected from patients enrolled at the Nucleus Network clinical site at the designated time points. The blood was stimulated with 10 ng/mL human IL-4 for 15 min, then, following lysis of the red blood cells and fixation of the leukocytes, staining for pSTAT6 and CD3 markers was performed and subsequently subjected to FACS analysis. The mean and standard deviation of % pSTAT6+ CD3 cells in the subjects during the time-course of the sampling are presented in FIG. 10. Inhibition of pSTAT6 was observed from Cohort 2 (delivered dose 6.00 mg) onwards. The results from the subjects in Cohort 2 and 3 (delivered doses 6.00 mg and 20.0 mg) demonstrated the highest inhibition of the % of the pSTAT6+ CD3 cells between 1 to 8 hours post inhalation on Day 10.

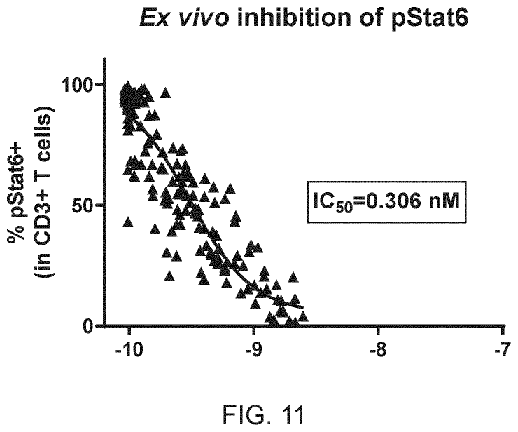

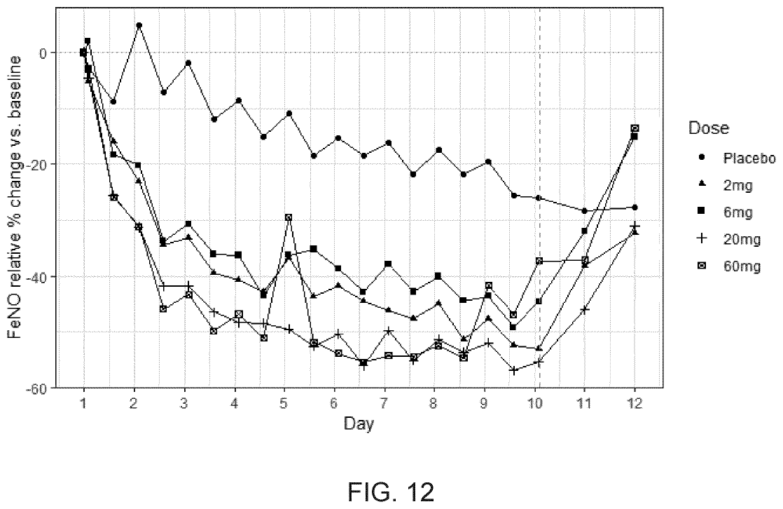

PK/PD analysis of the inhibition of ex vivo whole blood activation (FIG. 11) demonstrated a dose-dependent inhibition of the downstream STAT6 phosphorylation, with low variation between subjects, following inhalation of PRS-060/AZD1402. The IC₅₀ value was calculated at 0.306 nM.

(4) Safety Results For Cohort 1 (2.0 mg delivered dose) there were no clinically relevant changes observed in vital signs, electrocardiograms, pathology (biochemistry, haematology, urinalysis). In Cohort 2 (6.0 mg delivered dose) there was 1 subject whose neutrophil and white cell count increased from baseline to Day 10, there were no changes in vital signs or electrocardiograms in this cohort. In Cohort 3, 1 subject had an elevated white cell count deemed as not clinically significant, which normalised upon a repeat test, another subject had a haemaglobin drop that may have been related to repeated blood draws, there were no changes in vital signs or electrocardiograms in this cohort.

Mild to moderate adverse events were observed across the 3 cohorts. In Cohort 1 (delivered dose 2.0 mg) these included signs of a mild rash in 2 subjects, dry mouth post dosing in 1 subject. One subject experienced cough after dosing but this resolved before the next dose. In Cohort 2 (delivered dose 6.0 mg), 1 subject experienced dysgeusia, 1 subject experienced mild joint pain and 1 experienced cough that was mild in nature. In Cohort 3 (delivered dose 20.0 mg), headache and dry mouth were noted as being possibly related to Investigational Product, two episodes of bronchospasm were observed but were not related to dosing, short-term wheezing was seen in another 2 subjects and this was noted as possibly and probably related to dosing.

Overall, the investigational product was well tolerated and there were no concerns upon safety review that impacted the decision to dose escalate for all 3 cohorts.

### G. Discussion and Conclusions

In this multiple ascending dose study of PRS-060/AZD1402 in patients with mild asthma, a dose related systemic target engagement was observed in cohorts 1, 2 and 3 vs placebo, as represented by inhibition of STAT6 phosphorylation.

Overall, the reduction in FeNO indicated local target engagement of PRS-060/AZD1402 in the lung following inhalation. However, a key observation was the significant reduction of FeNO in subjects who received the 2 mg delivered dose (Cohort 1) that was not reflected in the systemic pSTAT6 target engagement, and at this twice daily delivered dose there was limited serum exposure insufficient for PK parameter calculation. This indicated a disconnect between the ability of PRS-060/AZD1402 to impact local lung inflammation as determined by a FeNO reduction without significant systemic exposure and target engagement. This provides support for the concept that a lung delivered lipocalin mutein targeting IL-4Rα can mediate anti-inflammatory effects without systemic exposure.

### Example 4. A Dose-Escalating, Single-Blind Study to Assess the Safety, Tolerability, and

Pharmacokinetics of Multiple Doses of PRS-060/AZD1402 Administered by Oral Inhalation in Subjects with Mild Asthma

Example 4 provides data for cohorts 1-5. Data for cohorts 1-3 was provided in Example 3. As the clinical trial has not yet completed the data lock for the overall clinical study, and final data outputs have not yet been produced for the study report.

### A. Study Objectives and Overview

The study objectives were as described in Example 3 above.

The baseline characteristics of the patients from cohorts 1˜4 are shown in Table 15 below.

TABLE 15
baseline characteristics

| | Placebo | | AZD1402/PRS-060 | | Overall | |
|---|---|---|---|---|---|---|
| Parameter | (N = 12) | | (N = 30) | | (N = 42) | |
| Age, years, mean (range) | 28.8 | (19-52) | 28.4 | (19-51) | 28.4 | (19-52) |
| Sex, male, n | 11 | | 26 | | 37 | |
| Race, n | | | | | | |
| White | 8 | | 25 | | 33 | |
| Asian/Pacific Islander | 2 | | 3 | | 5 | |
| Other | 2 | | 2 | | 4 | |
| BMI, kg/m², mean (range) | 27.7 | (22.5-34.8) | 25.0 | (20.5-33.4) | 25.7 | (20.5-34.8) |
| FeNO, ppb at pre-dose | 61.2 | (28-122) | 81.7 | (32-178) | 75.9 | (28-178) |
| day 1, mean (range) | | | | | | |
| FEV1, mL at pre-dose | 3730.8 | (2560-4770) | 3901.7 | (2580-5070) | 3852.9 | (2560-5070) |
| day 1, mean (range) | | | | | | |
| FEV1/FVC ratio, % at | 74.1 | (63-85) | 74.9 | (62-87) | 74.7 | (62-87) |
| predose | | | | | | |
| day 1, mean (range) | | | | | | |
| Patients from cohorts 1-4. | | | | | | |

BMI, body mass index; FeNO, fractional nitric oxide concentration in exhaled breath; FEV1, forced
expiratory volume in the first second; FVC, forced vital capacity; ppb, parts per billion.

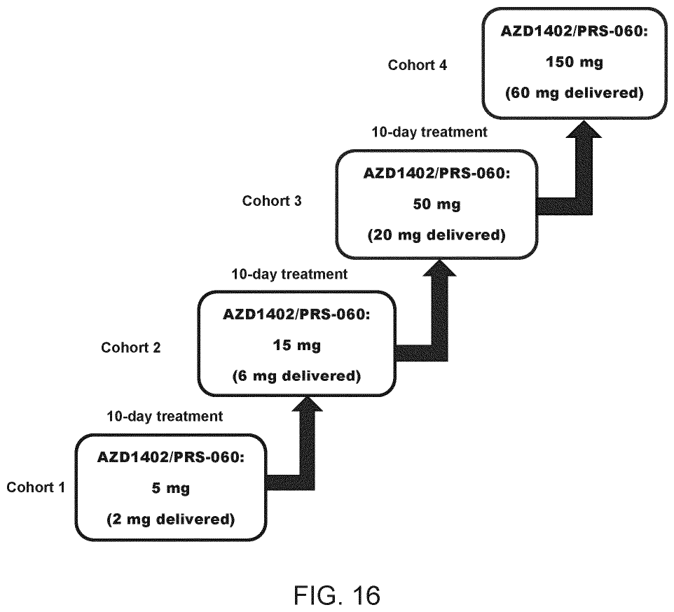

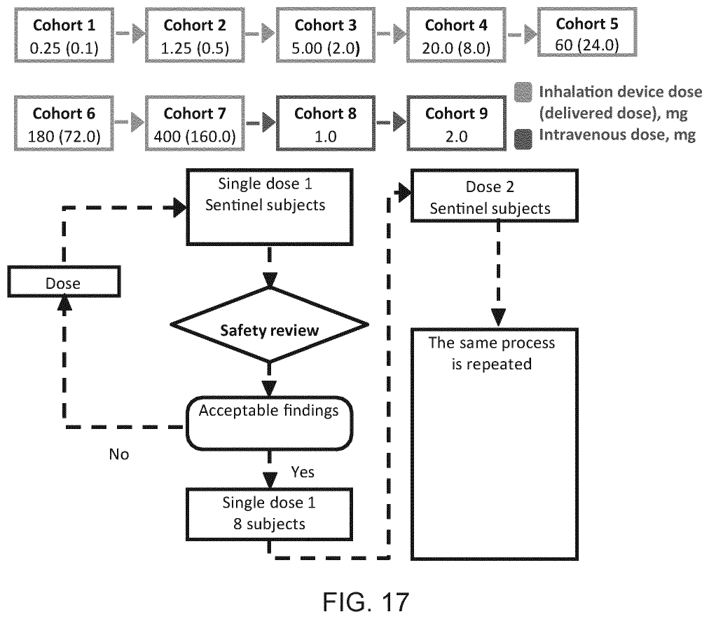

A schematic of the study design for cohorts 1 to 4 only is shown in FIG. 16.

### B. Study Procedures

The study procedures were as described in Example 3 above.

### C. Endpoints and Assessments

The endpoints and assessments were as described in Example 3 above.

### D. Data Analysis

(i) PK

For the secondary endpoints, PK profiles of PRS-060/AZD1402 on Day 1 and Day 10 for the first 5 cohorts were investigated during the course of the study and the PK population was used in data analyses. The exposure PK parameters were derived according to standard noncompartmental analytical procedures. The software used was Phoenix™ WinNonlin® v 8.0 (Pharsight Corporation, USA). Descriptive statistics of PK exposure parameters included arithmetic mean, and standard deviation (SD), per Table 19 and descriptive mean serum concentration versus time profiles were generated.

(ii) Anti-Drug Antibody Formation

The immunogenicity of PRS-060/AZD1402 (as assessed by anti-PRS-060/AZD1402 antibody formation) was investigated during the course of the study.

(iii) PD—FeNO

The assessment of PD marker FeNO was as described in Example 3 part (iii).

(iv) Analysis of FeNO

The non-linear mixed effect model described in Example 3 (iv) was updated by adding baseline FeNO as a covariate in the model of the asymptote parameter A.

### E. Results

(1) PK Results

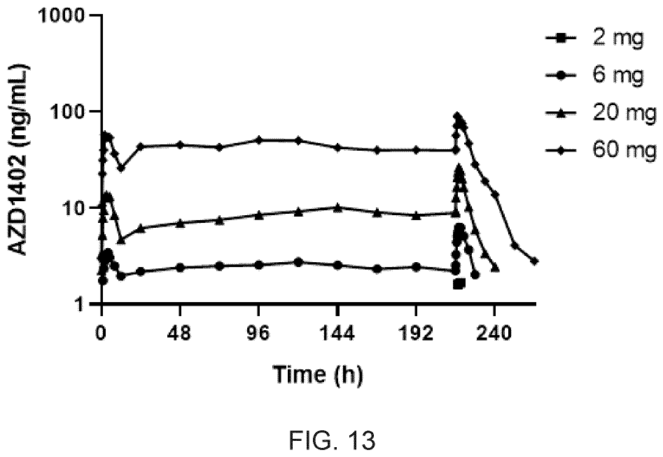

Limited serum exposure was observed after subjects received a delivered dose of 2 mg in Cohort 1 and this was insufficient for PK parameter calculation. More complete exposure data was observed after the subjects received delivered doses of 6 mg in Cohort 2, after 20 mg in Cohort 3, and 60 mg in cohort 4 allowing PK parameters to be derived (FIG. 13). The main findings from cohorts 1-5 were as follows:
AUC and Cₘₐₓ exposures increased with dose (Table 16).
Pre-dose samples were taken throughout the 10 day dosing period and the observed serum levels indicated that steady state had been achieved by the end of the 2nd day of dosing (FIG. 13)
Exposures after dosing on Day 10 following 9 days twice-daily administration were higher than after the 1st dose on Day 1. The increase observed was reasonably consistent with the dosing interval of 12 h and the PK properties derived in the single ascending dose study (Example 2).
No urinary PK analysis has been completed.

TABLE 16
Analysis exposure data on Day 1 and 10 of dosing

| Delivered | | | | |
|---|---|---|---|---|
| Doses of | | | | |
| PRS-060/ | PK parameter mean value (SD) | | | |
| AZD1402 | Cₘₐₓ (ng/mL) | | AUC₍₀₋₁₂₎ (h * ng/mL) | |
| (mg) | Day 1 | Day 10 | Day 1 | Day 10 |
| 0.2 | Not | Not | Not | Not |
| | determined* | determined* | determined* | determined* |
| 2 | Not | Not | Not | Not |
| | determined* | determined* | determined* | determined* |
| 6 | 5.00 (3.39) | 6.33 (1.63) | 38.5 (21.4) | 54.0 (13.9) |
| 20 | 15.2 (7.39) | 27.8 (13.0) | 122 (65.0) | 210 (97.6) |
| 60 | 69.6 (38.6) | 103 (31.1) | 713 (395) | 780 (340) |

*Not determined due to lack of subjects with measurable exposures

(3) Anti-Drug Antibodies

In Cohort 1, of the 6 subjects, 2 returned a low positive ADA value on Day 17; subsequent responses on Day 40 were negative.

In Cohort 2 there were no ADA findings.

In Cohort 3, of the 12 subjects, 4 returned a low positive ADA value on Day 40, and 1 subject on Day 17 with a subsequent negative response at Day 40.

In Cohort 4, of the 6 subjects, 3 returned a positive ADA value. Of these 2 subjects had a low positive ADA value on Day 40 only. 1 subject yielded a higher positive ADA response on both Day 17 and Day 40.

In Cohort 5, there was one subject with confirmed positive ADA results on Day 12, 17 & 40 and 1 subject with a confirmed positive ADA on Day 40.

No ADA were observed in any pretreatment samples or in those whilst receiving PRS-060/AZD1402.

(4) Exploratory Endpoint—Target Engagement by Monitoring pSTAT6 Inhibition in CD3+ Cells:

Inhibition of STAT6 phosphorylation was evaluated to assess PRS-060/AZD1402 target engagement.

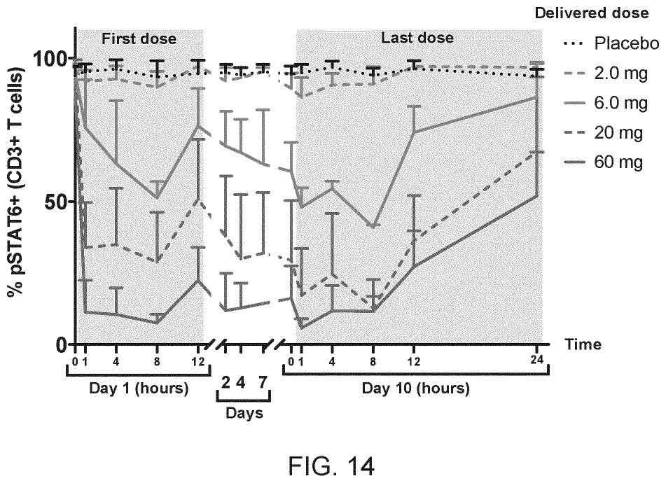

Ex-vivo whole blood stimulation with IL-4 was performed in blood of subjects enrolling in one of the sites from Cohorts 1 to 4 but not Cohort 5, and the corresponding pSTAT6 levels were determined. Whole blood was collected from patients enrolled at the Nucleus Network clinical site at the designated time points. The blood was stimulated with 10 ng/mL human IL-4 for 15 min, then, following lysis of the red blood cells and fixation of the leukocytes, staining for pSTAT6 and CD3 markers was performed and subsequently subjected to FACS analysis. The mean and standard deviation of % pSTAT6+ CD3 cells in the subjects during the time-course of the sampling are presented in FIG. 14. Inhibition of pSTAT6 was observed from Cohort 2 (delivered dose 6.00 mg) onwards. The results from the subjects in Cohort 3 and 4 (delivered doses 20.00 mg and 60.0 mg) demonstrated the highest inhibition of the % of the pSTAT6+ CD3 cells between 1 to 8 hours post inhalation on Day 10.

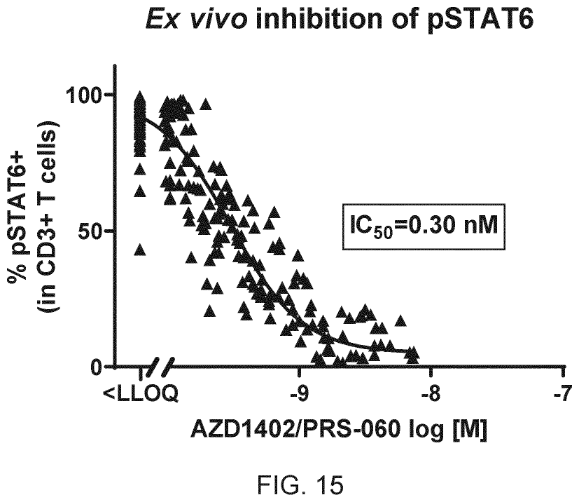

PK/PD analysis of the inhibition of ex vivo whole blood activation (FIG. 15) demonstrated a dose-dependent inhibition of the downstream STAT6 phosphorylation, with low variation between subjects, following inhalation of PRS-060/AZD1402. The IC₅₀ value was calculated at 0.30 nM.

(5) Safety Results: Cohorts 1-5

Adverse Events: Mild to moderate adverse events attributable to the drug were observed across the 5 cohorts, summarised as follows:

In Cohort 1 (twice daily delivered dose 2.0 mg) adverse events included signs of a mild rash in 2 subjects, dry mouth post dosing in 1 subject. One subject experienced cough after dosing but this resolved before the next dose.

In Cohort 2 (twice daily delivered dose 6.0 mg), adverse events included, 1 subject experienced dysgeusia, 1 subject experienced mild joint pain and 1 experienced a mild cough and a subsequent short, and mild asthma exacerbation 2 days after the end of dosing.

In Cohort 3 (twice daily delivered dose 20.0 mg), adverse events included, headache and dry mouth noted as being possibly related to Investigational Product. Two events of bronchospasm were observed in a single subject but were not related to dosing. Short-term wheezing was seen in another 2 subjects and noted as possibly and probably related to dosing respectively.

In Cohort 4 (twice daily delivered dose 60.0 mg), adverse events included, headache as possibly related to Investigational Product. Two episodes of coughing and associated symptoms also noted as possibly to definitely related to Investigational Product. On day 9, one subject diagnosed with an upper respiratory tract infection, experienced a brief bronchospasm. One subject was withdrawn from the study on Day 9 with a cough and high fevers, considered an upper respiratory tract infection deemed probably related to the drug. This participant also had significant episodes of syncope on multiple occasions which is likely related to the viral illness. The same participant also had one adverse event of cold sores (ongoing) and chest tightness post the dosing period, likely related to the viral illness. Additionally, this subject, on return for their post dosing follow up visit was confirmed with an unrelated pregnancy. The subsequent outcome of the pregnancy was a miscarriage, not related to Investigational Product and likely due to the participant's age (47 years).

Additional safety summarised as follows:

For Cohort 1 (2.0 mg twice daily delivered dose) there were no clinically relevant changes observed in vital signs, electrocardiograms, pathology (biochemistry, haematology, urinalysis).

In Cohort 2 (6.0 mg twice daily delivered dose) there was 1 subject whose neutrophil and white cell count increased from baseline to Day 10. There were no changes in vital signs or electrocardiograms in this cohort.

In Cohort 3 (20.0 mg twice daily delivered dose), 1 subject had an elevated white cell count deemed as not clinically significant, which normalised upon a repeat test, another subject had a haemoglobin drop that may have been related to repeated blood draws. There were no changes in vital signs or electrocardiograms in this cohort.

In Cohort 4 (60.0 mg twice daily delivered dose), one participant had a low neutrophil count prior to dosing and not related to the Investigational Product. There was also a case of mild transient lymphopenia and neutropenia, not considered to be significant. One subject showed haematology fluctuations with a drop in haemoglobin and the post treatment follow result was consistent with their day 1 result. One subject was observed to have a pre-dose elevated bilirubin (normal at screening) which continued to increase during the study, but decreased at an unscheduled visit. However, it still remains high and the adverse event is ongoing.

In Cohort 5 (0.2 mg twice daily delivered dose), 26 mild to moderate adverse events were observed in 9 of the 11 subjects enrolled. None of the adverse events were considered serious or severe. 20 of the 26 AEs were considered “mild,” 5 of which were considered “possibly related.” 6 adverse events were considered moderate in nature, 3 of which were considered “possibly related” which were observed in one subject who received placebo. The remaining 3 moderate AEs were considered “not related.” All AEs observed in Cohort 5 resolved. All spirometry, laboratory, ECG, and vital signs were considered not clinically significant by the clinical Investigators. The safety review committee decided unanimously that the dose level was well tolerated.

Overall, the investigational product was well tolerated in this study and there were no concerns upon safety review that impacted the decision to dose escalate and/or continue to the next cohort for all 5 cohorts.

TABLE 17
provides a summary of adverse events from cohorts 1-4,
which occurred in 5% of overall patients^{a}

| | | AZD1402/ | |
|---|---|---|---|
| | | PRS-060^{c} | Overall |
| System organ class | Placebo | (N = 30) | (N = 42) |
| AE Preferred Terms^{b} | n (%) m | n (%) m | n (%) m |
| Gastrointestinal disorders | 4 (33.3) 4 | 13 (43.4) 14 | 17 (40.5) 18 |
| Dry mouth | 1 (8.3) 1 | 2 (6.7) 2 | 3 (7.1) 3 |
| Nausea | 1 (8.3) 1 | 3 (10.0) 3 | 4 (9.5) 4 |
| Infections and infestations | 1 (8.3) 1 | 7 (23.3) 8 | 8 (19.0) 9 |
| Upper respiratory tract infection | 1 (8.3) 1 | 3 (10.0) 4 | 4 (9.5) 5 |
| Nervous system disorders | 5 (41.7) 9 | 13 (43.4) 18 | 18 (42.9) 27 |
| Headache | 3 (25.0) 6 | 5 (16.7) 7 | 8 (19.0) 13 |
| Presyncope | 0 | 4 (13.3) 6 | 4 (9.5) 6 |
| Respiratory, thoracic and | 6 (50.0) 6 | 14 (46.7) 15 | 20 (47.6) 21 |
| mediastinal disorders | | | |
| Cough | 1 (8.3) 1 | 4 (13.3) 4 | 5 (11.9) 5 |
| Rhinorrhoea | 2 (16.7) 2 | 1 (3.3) 1 | 3 (7.1) 3 |
| Wheezing | 2 (16.7) 2 | 4 (13.3) 5 | 6 (14.3) 7 |

^{a}Percentage is based on Preferred Term i.e, the incidence of AEs which occurred in ≥5%, of overall patients by preferred term
^{b}AEs are from cohorts 1-4, which occurred in ≥5% of overall patients
^{c}Delivered doses of AZD1402/PRS-060 were 2 mg, 6 mg, 20 mg and 60 mg
One pregnancy leading to a serious AE of miscarriage was observed. This was considered to be due to the patient's age, and not related to the study drug by the investigator
AE, adverse event; m, number of events; n, number of patients reported with specific AEs; N, total number of patients in each treatment group

(6) Cohort 1-5: FeNO Results

FeNO baseline mean (SD) across cohorts 1-4 (n=42) was 75.8 (41.2) ppb, median was 62 ppb and the range was 28-178 ppb. The updated non-linear mixed effect model, including baseline FeNO as a covariate was used to estimate the mean percent reduction. The estimated percent reduction in placebo group (n=12) after 10 days of treatment was 26.2%.

TABLE 18
Mean percent reduction in FeNO from baseline relative to placebo.
Estimated treatment effects represent the reduction at end of
treatment (Day 10).

| Delivered | | | | |
|---|---|---|---|---|
| Doses of PRS- | | LS mean | | |
| 060/AZD1402 | Number | reduction vs | | |
| and Matching | of | baseline, % | Percent reduction vs | |
| Placebo (mg) | patients | (95% CI) | placebo (95% CI) | P-value* |
| 60 mg | 6 | 48.7 (37-58) | 30.5% (10%, 46%) | 0.005 |
| 20 mg | 12 | 53.1 (46-59) | 36.4% (22%, 48%) | <0.0001 |
| 6 mg | 6 | 44.1 (31-54) | 24.3% (2.7%, 41%) | 0.03 |
| 2 mg | 6 | 43.9 (31-54) | 24.0% (1.8%, 41%) | 0.04 |
| Placebo | 12 | 26.2 (14-36) | | |

*Two-sided test of null hypothesis “no difference between active and placebo”

Analysis of the FeNO data for Cohort 5, delivered dose 0.2 mg, indicated no reduction of FeNO from baseline relative to placebo.

### G. Discussion and Conclusions

In this multiple ascending dose study of PRS-060/AZD1402 in patients with mild asthma, a dose related systemic target engagement was observed in cohorts 1-5 (i.e. delivered doses of 2 mg, 6 mg, 20 mg, 60 mg and 0.2 mg) vs placebo, as represented by inhibition of STAT6 phosphorylation.

Overall, the reduction in FeNO indicated local target engagement of PRS-060/AZD1402 in the lung following inhalation. However, a key observation was the significant reduction of FeNO in subjects who received the 2 mg delivered dose (Cohort 1) that was not reflected by a significant inhibitory effect in the systemic pSTAT6 target engagement assay as, at this twice daily delivered dose, the limited systemic exposure seen was insufficient to inhibit this IL-4 induced response.

This indicated a disconnect between the demonstrated ability of PRS-060/AZD1402 to impact local lung inflammation as determined by a FeNO reduction but without detectable systemic exposure and associated systemic target engagement. This provides support for the concept that a lung delivered lipocalin mutein targeting IL-4Rα can mediate anti-inflammatory effects without detectable systemic exposure.

TABLE 19
Pharmacokinetic Parameters

| Parameter | Explanation |
|---|---|
| AUC_{0-τ} | Area under the curve from time zero to the end of the |
| | dosing period |
| AUC₀₋₁₂ | Serum AUC from time zero to 12 hours post-dose |
| AUC₀₋₂₄ | Serum AUC from time zero to 24 hours post-dose |
| AUC₀₋ₗₐₛₜ | Serum AUC from time zero to the last measurable |
| | concentration sampling time (tlast) (mass × time × |
| | volume-1) |
| AUC_{inf} | Serum AUC from time zero to infinity (mass × time × |
| | volume-1) |
| AUC₀₋₂₄/Dose | Dose-normalized serum AUC from time zero to 24 |
| | hours post-dose |
| AUC₀₋ₗₐₛₜ/Dose | Dose-normalized serum AUC from time zero to the |
| | last measurable concentration sampling time (tₗₐₛₜ) |
| | (mass × time × volume-1) |
| AUC_{inf}/Dose | Dose-normalized serum AUC from time zero to |
| | infinity (mass × time × volume-1) |
| Rac AUC_{0-τ} | Accumulation ratio for AUC_{0-τ} estimated as AUC_{(0-τ)} |
| | Day 10/AU0C₀₋₁₂ on the final (10^{th}) day of dosing if |
| | extrapolated part is less than 20% |
| Rac Cₘₐₓ | Accumulation ratio for Cₘₐₓ, estimated as Cₘₐₓ |
| | Day 10/Cₘₐₓ Day 1 |
| TCP | Temporal change parameter |
| Cₐᵥₑ | Average serum concentration during a dosing interval |
| | estimated as AUC_{0-τ}/12 |
| Cₘₐₓ | The maximum (peak) observed blood, serum, or other |
| | body fluid drug concentration (mass × volume-1) |
| Cₘₐₓ/Dose | Dose-normalized maximum (peak) observed blood, |
| | serum, or other body fluid drug concentration (mass × |
| | volume-1) |
| Tₘₐₓ | The time to reach maximum (peak) blood, serum, or |
| | other body fluid drug concentration after dosing (time) |
| t_{1/2} | The elimination half-life associated with the terminal |
| | slope (λz) of a semi-logarithmic concentration-time |
| | curve (time). Use qualifier for other half-lives |
| CL/F | The apparent total body clearance of drug from the |
| | serum following inhalation (volume × time-1). |
| Vz/F | The apparent volume of distribution based on the |
| | terminal phase. |
| Ae | The total amount of drug excreted in urine over the |
| | entire collection interval (i.e. from 0 to 48 hours |
| | post-dose). |
| Ae(tₓ-tₓ₊₁₎ | Amount of drug excreted unchanged in the urine over |
| | time interval tₓ to tₓ₊₁ Calculated for each collection |
| | interval |
| Ae(0-tₓ) | Cumulative amount of drug excreted in the urine over |
| | time interval 0 to tₓ. Calculated for each collection |
| | interval |
| fe | The fraction of dose excreted in urine over the entire |
| | collection interval (i.e. from 0 to 48 hours post-dose) |
| fe(tₓ-tₓ₊₁) | Fraction of dose excreted unchanged in urine over time |
| | interval tₓ to tₓ₊₁ |
| | Calculated for each collection interval |
| fe(0-tₓ) | Cumulative fraction of dose excreted unchanged in |
| | urine over time interval 0 to tₓ |
| | Calculated for each collection interval. |
| CLr | The renal clearance |
| Tₗₐₛₜ | The last measurable concentration sampling time |
| MRT | Mean residence time |
| MRT_{inf} | Mean residence time extrapolated to infinity |
| V_{z} | Volume of distribution at terminal phase |
| Vₛₛ | Volume of distribution at steady state |
| CL | Systemic clearance of drug from the plasma/serum |
| F_{inhalation, total} | Absolute systemic bioavailability after inhalation |
| MAT | Mean absorption time |
| FEV₁ | Forced expiratory volume in 1 second |
| FEV₆ | Forced expiratory volume in 6 seconds |
| FVC | Forced vital capacity |
| PEFR | Peak expiratory flow rate |
| FEV₁/FVC | The amount of air exhaled forcefully in one second |
| ratio | (FEV1) compared to the full amount of air that can be |
| | forcefully exhaled in a complete breath |
| RR interval | The distance between two consecutive R waves |
| PR interval | The period, measured in milliseconds, that extends |
| | from the beginning of the P wave (the onset of atrial |
| | depolarization) until the beginning of the QRS |
| | complex (the onset of ventricular depolarization) |
| QT interval | A measure of the duration of ventricular repolarization |
| QTc | Corrected QT |
| QTcB interval | QTc corrected by Bazett's formula |
| QTcF interval | QTc corrected by Fridericia's formula |

### REFERENCES

A number of publications are cited above in order to more fully describe and disclose the invention and the state of the art to which the invention pertains. Full citations for these references are provided below. The entirety of each of these references is incorporated herein.
Pervaiz, S., & Brew, K. (1987) FASEB J. 1, 209-214
Flower, D. R. (1996) Biochem. J. 318, 1-14
Flower, D. R. et al. (2000) Biochim. Biophys. Acta 1482, 9-24
Skerra, A. (2000) Biochim. Biophys. Acta 1482, 337-350
You, J., et al. (2010) Electrophoresis 31, 1853-1861
Skerra, A. (2001) Rev. Mol. Biotechnol. 74, 257-275
Schlehuber, S., and Skerra, A. (2002) Biophys. Chem. 96, 213-228
Redl, B. (2000) Biochim. Biophys. Acta 1482; 241-248
Glasgow, B. J. et al. (1995) Curr. Eye Res. 14, 363-372
Gasymov, O. K. et al. (1999) Biochim. Biophys. Acta 1433, 307-320
Breustedt, D. A. et al. (2005) J. Biol. Chem. 280, 1, 484-493
Altschul, et al. (1997) Nucleic Acids Res. 25, 3389-3402
Altschul, et al. (1990) J. Mol. Biol. 215, 403-410
Smith, et al. (1981) J. Mol. Biol. 147, 195-197
Nelms et al., Annu Rev Immunol, 1999, 17:701-738
Chen et al., 2003. J Immunol, 171:3627-3635
Wirnsberger et al., Eur J Immunol., 2006, 36(7):1882-1891
Rahal et al., Int J Radiat Oncol Biol Phys, 2018, 100(4): 1034-1043
Hoeck and Woisetsschläger, J Immunol, 2001, 167(6):3216-3222
Pearson and Lipman, Proc. Natl. Acad. Sci. USA, 1988, 85(8):2444-2448

### SEQUENCES

TABLE 20

| SEQ ID NO: | Description | Protein/DNA | Sequence |
|---|---|---|---|
| 1 | Amino acid | Protein | ASDEEIQDVSGTVVYLKAMTVDSRCPRAV |
| | sequence of | | YNSVTPMTLTTLEGGNLEAKFTAQRKGR |
| | the human tear | | WQKYKLVLEKTDEPGKYTASGGRHVAYII |
| | lipocalin | | RSHVKDHYIFHSEGLCPGQPVPGVWLVG |
| | mutein PRS- | | RDPKNNLEALEDFEKAAGARGLSTESILIP |
| | 060/AZD1402 | | RQSETSSPGSD |
| 2 | Human tear | Protein | MKPLLLAVSLGLIAALQAHHLLASDEEIQD |
| | lipocalin | | VSGTVVYLKAMTVDREFPEMNLESVTPMT |
| | | | LTTLEGGNLEAKVTMLISGRCQEVKAVLE |
| | | | KTDEPGKYTADGGKHVAYIIRSHVKDHYIF |
| | | | YCEGELHGKPVRGVKLVGRDPKNNLEAL |
| | | | EDFEKAAGARGLSTESILIPRQSETCSPGS |
| | | | D |
| 3 | Mature form of | Protein | HHLLASDEEIQDVSGTVVYLKAMTVDREFP |
| | human tear | | EMNLESVTPMTLTTLEGGNLEAKVTMLIS |
| | lipocalin | | GRCQEVKAVLEKTDEPGKYTADGGKHVA |
| | | | YIIRSHVKDHYIFYCEGELHGKPVRGVKLV |
| | | | GRDPKNNLEALEDFEKAAGARGLSTESILI |
| | | | PRQSETCSPGSD |
| 4 | Amino acid | Protein | MKVLQEPTCVSDYMSISTCEWKMNGPTN |
| | sequence of | | CSTELRLLYQLVFLLSEAHTCIPENNGGAG |
| | human | | CVCHLLMDDVVSADNYTLDLWAGQQLLW |
| | interleukin-4 | | KGSFKPSEHVKPRAPGNLTVHTNVSDTLL |
| | receptor alpha | | LTWSNPYPPDNYLYNHLTYAVNIWSENDP |
| | chain | | ADFRIYNVTYLEPSLRIAASTLKSGISYRAR |
| | | | VRAWAQCYNTTWSEWSPSTKWHNSYRE |
| | | | PFEQHLLLGVSVSCIVILAVCLLCYVSITKIK |
| | | | KEVWVDQIPNPARSRLVAIIIQDAQGSQWE |
| | | | KRSRGQEPAKCPHWKNCLTKLLPCFLEH |
| | | | NMKRDEDPHKAAKEMPFQGSGKSAWCP |
| | | | VEISKTVLWPESISVVRCVELFEAPVECEE |
| | | | EEEVEEEKGSFCASPESSRDDFQEGREGI |
| | | | VARLTESLFLDLLGEENGGFCQQDMGES |
| | | | CLLPPSGSTSAHMPWDEFPSAGPKEAPP |
| | | | WGKEQPLHLEPSPPASPTQSPDNLTCTE |
| | | | TPLVIAGNPAYRSFSNSLSQSPCPRELGP |
| | | | DPLLARHLEEVEPEMPCVPQLSEPTTVPQ |
| | | | PEPETWEQILRRNVLQHGAAAAPVSAPTS |
| | | | GYQEFVH |
| | | | AVEQGGTQASAVVGLGPPGEAGYKAFSS |
| | | | LLASSAVSPEKCGFGASSGEEGYKPFQDL |
| | | | IPGCPGDPAPVPVPLFTFGLDREPPRSPQ |
| | | | SSHLPSSSPEHLGLEPGEKVEDMPKPPLP |
| | | | QEQATDPLVDSLGSGIVYSALTCHLCGHL |
| | | | KQCHGQEDGGQTPVMASPCCGCCCGDR |
| | | | SSPPTTPLRAPDPSPGGVPLEASLCPASL |
| | | | APSGISEKSKSSSSFHPAPGNAQSSSQTP |
| | | | KIVNFVSVGPTYMRVS |
| 5 | Amino acid | Protein | MKVLQEPTCVSDYMSISTCEWKMNGPTN |
| | sequence of | | CSTELRLLYQLVFLLSEAHTCIPENNGGAG |
| | extracellular | | CVCHLLMDDVVSADNYTLDLWAGQQLLW |
| | domain of | | KGSFKPSEHVKPRAPGNLTVHTNVSDTLL |
| | human | | LTWSNPYPPDNYLYNHLTYAVNIWSENDP |
| | interleukin-4 | | ADFRIYNVTYLEPSLRIAASTLKSGISYRAR |
| | receptor alpha | | VRAWAQCYNTTWSEWSPSTKWHNSYRE |
| | chain | | PFEQH |

1. A method for treating asthma in a human subject, wherein the method comprises administering by inhalation a therapeutically effective amount of an anti-IL-4 receptor alpha (IL-4Rα) lipocalin mutein comprising the amino acid sequence set forth in SEQ ID NO: 1, or a variant or fragment thereof, to said subject at least once per day, wherein said lipocalin mutein, or variant or fragment thereof, is delivered at a dose of about 0.1 mg to about 160 mg.

2. The method of claim 1, wherein the delivered dose of said lipocalin mutein, or variant or fragment thereof, is at least about 8 mg.

3. The method of claim 2, wherein the delivered dose results in systemic exposure of said lipocalin mutein, or variant or fragment thereof.

4. The method of claim 2, wherein administering said lipocalin mutein, or variant or fragment thereof, to said subject results in inhibition of IL-4 stimulated STAT6 phosphorylation in CD3+ T cells in said subject.

5. The method of claim 4, wherein administering said lipocalin mutein, or variant or fragment thereof, results in at least about 10%, at least about 20%, at least about 30%, at least about 40%, at least about 50%, at least about 60%, at least about 70%, at least about 80%, at least about 90%, at least about 95%, or at least about 99% inhibition of IL-4 stimulated STAT6 phosphorylation in CD3+ T cells in the subject.

6. The method of claim 2, wherein administering said lipocalin mutein, or variant or fragment thereof, results in inhibition of IL-4 stimulated STAT6 phosphorylation in CD3+ T cells with an IC₅₀ of about 10 nM or lower.

7. The method of claim 1, wherein the delivered dose of the lipocalin mutein, or variant or fragment thereof, is less than about 2 mg.

8. The method of claim 1, wherein a fractional nitric oxide concentration in exhaled breath (FeNO) is reduced following administration of said lipocalin mutein or variant or fragment thereof to said subject.

9. The method of claim 8, wherein FeNO is reduced by at least 10%, by at least 15%, by at least 20%, by at least 25%, by at least 30%, by at least 35%, by at least 40%, by at least 45% or by at least 50% following administration of said lipocalin mutein or variant or fragment thereof to said subject.

10. The method of claim 1, wherein said lipocalin mutein, or variant or fragment thereof, is administered to the subject by nebulization.

11. The method of claim 1, wherein the delivered dose of said lipocalin mutein, or variant or fragment thereof, is from about 0.2 mg to about 60 mg.

12. The method of claim 1, wherein the delivered dose of said lipocalin mutein, or variant or fragment thereof, is at least about 6 mg.

13. The method of claim 12, wherein the delivered dose of said lipocalin mutein, or variant or fragment thereof, of at least about 6 mg is administered twice daily.

14. The method of claim 12, wherein the delivered dose results in systemic exposure of said lipocalin mutein, or variant or fragment thereof.

15. The method of claim 12, wherein administering said lipocalin mutein, or variant or fragment thereof, to said subject results in inhibition of IL-4 stimulated STATE phosphorylation in CD3+ T cells in said subject.

16. The method of claim 15, wherein administering said lipocalin mutein, or variant or fragment thereof, results in at least about 10%, at least about 20%, at least about 30%, at least about 40%, at least about 50%, at least about 60%, at least about 70%, at least about 80%, at least about 90%, at least about 95%, or at least about 99% inhibition of IL-4 stimulated STAT6 phosphorylation in CD3+ T cells in the subject.

17. The method of claim 12, wherein administering said lipocalin mutein, or variant or fragment thereof, results in inhibition of IL-4 stimulated STAT6 phosphorylation in CD3+ T cells with an IC₅₀ of about 10 nM or lower.

18. The method of claim 1, wherein the delivered dose of the lipocalin mutein, or variant or fragment thereof, is about 2 mg or less.

19. The method of claim 18, wherein the delivered dose of the lipocalin mutein, or variant or fragment thereof, of about 2 mg or less is administered twice daily.

20. The method of claim 11, wherein the delivered dose results in local lung exposure of said lipocalin mutein, or variant or fragment thereof.

21. The method of claim 11, wherein a fractional nitric oxide concentration in exhaled breath (FeNO) is reduced following administration of said lipocalin mutein or variant or fragment thereof to said subject.

22. The method of claim 21, wherein FeNO is reduced by at least 10%, by at least 15%, by at least 20%, by at least 25%, by at least 30%, by at least 35%, by at least 40%, by at least 45% or by at least 50% following administration of said lipocalin mutein or variant or fragment thereof to said subject.

23. The method of claim 11, wherein said lipocalin mutein, or variant or fragment thereof, is administered to the subject by nebulization.

24.-69. (canceled)


SEQUENCE LISTING (5 sequences)

SEQ ID NO: 1 - protein, 154 aa, Artificial Sequence
  Amino acid sequence of the human tear lipocalin mutein PRS-060/AZD1402
ASDEEIQDVSGTWYLKAMTVDSRCPRAVYNSVTPMTLTTLEGGNLEAKFTAQRKGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPGSD

SEQ ID NO: 2 - protein, 176 aa, Artificial Sequence
  Human tear lipocalin
MKPLLLAVSLGLIAALQAHHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 3 - protein, 158 aa, Artificial Sequence
  Mature form of human tear lipocalin
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 4 - protein, 800 aa, Artificial Sequence
  Amino acid sequence of human interleukin-4 receptor alpha chain
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 5 - protein, 207 aa, Artificial Sequence
  Amino acid sequence of extracellular domain of human interleukin-4 receptor alpha chain
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH